SEQUENCE LISTING (28 sequences)

SEQ ID NO: 1 - DNA, 21 bp, synthetic construct
  source 1..21: mol_type = other DNA
gcctgcatcaatatttctcat

SEQ ID NO: 2 - DNA, 21 bp, synthetic construct
  source 1..21: mol_type = other DNA
gtttcagcagaaggagtctta

SEQ ID NO: 3 - DNA, 21 bp, synthetic construct
  source 1..21: mol_type = other DNA
ggaggaatccaacaggtcaaa

SEQ ID NO: 4 - DNA, 21 bp, synthetic construct
  source 1..21: mol_type = other DNA
gtcgggcatttaagcaacata

SEQ ID NO: 5 - DNA, 21 bp, synthetic construct
  source 1..21: mol_type = other DNA
gcatggaaccaacgacaatga

SEQ ID NO: 6 - DNA, 21 bp, synthetic construct
  source 1..21: mol_type = other DNA
gaaagacaagacgctgattga

SEQ ID NO: 7 - DNA, 21 bp, synthetic construct
  source 1..21: mol_type = other DNA
gcaaagatccaagactatcat

SEQ ID NO: 8 - DNA, 21 bp, synthetic construct
  source 1..21: mol_type = other DNA
gggcagaattagcaaacctta

SEQ ID NO: 9 - DNA, 21 bp, synthetic construct
  source 1..21: mol_type = other DNA
gaacagaaatacacctacgaa

SEQ ID NO: 10 - DNA, 21 bp, synthetic construct
  source 1..21: mol_type = other DNA
cgacagtatgatgaacacagt

SEQ ID NO: 11 - DNA, 21 bp, synthetic construct
  source 1..21: mol_type = other DNA
cctctgtctatggagacttta

SEQ ID NO: 12 - DNA, 21 bp, synthetic construct
  source 1..21: mol_type = other DNA
gcgtgtcttcacagatctgaa

SEQ ID NO: 13 - DNA, 21 bp, synthetic construct
  source 1..21: mol_type = other DNA
ccaccttatcttagagtcatt

SEQ ID NO: 14 - DNA, 21 bp, synthetic construct
  source 1..21: mol_type = other DNA
tcagcgatagctatgacattt

SEQ ID NO: 15 - DNA, 25 bp, synthetic construct
  source 1..25: mol_type = other DNA
caccgtgtacgagtcggtggccagc

SEQ ID NO: 16 - DNA, 25 bp, synthetic construct
  source 1..25: mol_type = other DNA
caccgagtaaggaaggtgaaatacg

SEQ ID NO: 17 - DNA, 21 bp, synthetic construct
  source 1..21: mol_type = other DNA
cagcagcagaacacagtacca

SEQ ID NO: 18 - DNA, 21 bp, synthetic construct
  source 1..21: mol_type = other DNA
ctgaccacagatacaggcact

SEQ ID NO: 19 - DNA, 20 bp, synthetic construct
  source 1..20: mol_type = other DNA
gaggatgaggtggaacgtgt

SEQ ID NO: 20 - DNA, 20 bp, synthetic construct
  source 1..20: mol_type = other DNA
agaagtgacgcagccctcta

SEQ ID NO: 21 - DNA, 19 bp, synthetic construct
  source 1..19: mol_type = other DNA
gtgtcgcaccctgtcaaag

SEQ ID NO: 22 - DNA, 22 bp, synthetic construct
  source 1..22: mol_type = other DNA
ggggacaagacctaattgaacc

SEQ ID NO: 23 - DNA, 20 bp, synthetic construct
  source 1..20: mol_type = other DNA
gcaaccacgcatgagaactg

SEQ ID NO: 24 - DNA, 19 bp, synthetic construct
  source 1..19: mol_type = other DNA
ctttgacagggtgcgacac

SEQ ID NO: 25 - DNA, 20 bp, synthetic construct
  source 1..20: mol_type = other DNA
ggggcatctgctcatttggt

SEQ ID NO: 26 - DNA, 21 bp, synthetic construct
  source 1..21: mol_type = other DNA
aagccgttatggtactgtgct

SEQ ID NO: 27 - DNA, 20 bp, synthetic construct
  source 1..20: mol_type = other DNA
acatcgacctcaccaagagg

SEQ ID NO: 28 - DNA, 20 bp, synthetic construct
  source 1..20: mol_type = other DNA
tcccatccttcacatccttc

(19) United States
(12) Patent Application Publication
(10) Pub. No.: US 2026/0034213 A1
(43) Pub. Date: Feb. 5, 2026

(54) SYSTEMS AND METHODS FOR TARGETING NAD-BASED PATHWAYS FOR TREATMENT OF CANCER

(71) Applicants: The University of Hong Kong, Hong Kong (CN); Centre for Oncology and Immunology Limited, Hong Kong (CN)

(72) Inventors: Carmen Chak Lui Wong, Hong Kong (CN); Wing-Sum Cheu, Hong Kong (CN); Kin Leung Kwan, Hong Kong (CN)

(21) Appl. No.: 19/289,764

(22) Filed: Aug. 4, 2025

Related U.S. Application Data
(60) Provisional application No. 63/678,922, filed on Aug. 2, 2024.

(51) Int. Cl. A61K 39/395; A61K 31/4409; A61K 31/4439; A61K 31/4545; A61K 31/47; A61K 31/7105; A61P 35/00; C12N 15/113; C12N 15/115

(52) U.S. Cl. CPC A61K 39/39558; A61K 31/4409; A61K 31/4439; A61K 31/4545; A61K 31/47; A61K 31/7105; A61P 35/00; C12N 15/1137; C12N 15/115; C12Y 204/02012; C12N 2310/11; C12N 2310/14; C12N 2310/141; C12N 2310/16

(57) ABSTRACT

It was discovered that ICIs elevated extracellular NAD levels and conferred cancer cells resistance to ICIs. It was also discovered that NAMPT was induced by IFN-y and ICIs resulting in an increase of extracellular NAD level, which could be reduced by NAMPT knockdown. NAMPT deficiency in HCC sensitized tumors to anti-PD-1 treatment. It was further discovered that extracellular NAD promoted T cell apoptosis, exhaustion, and T cell differentiation to regulatory T cells. The blockade of P2X7R reversed the immunosuppressive effect of NAD and worked effectively in combination with immunotherapies, indicating that it can be a combination therapeutic approach for ICI-resistant tumors. It was shown that serum NAD level can be a predictive biomarker for ICI responses in HCC. Overall, extracellular NAD was shown to act as an immunosuppressive metabolite that could serve as a biomarker for ICI responses and the P2X7R can be a therapeutic target to improve ICI efficacy.

### CROSS REFERENCE TO RELATED APPLICATION

This application claims benefit of and priority to U.S. Provisional Application No. 63/678,922, filed Aug. 2, 2024, which is specifically incorporated by reference herein in its entirety.

### REFERENCE TO THE SEQUENCE LISTING

The Sequence Listing submitted as an XML filed named “UHK_01530US_ST26.xml”, created on Jul. 29, 2025, and having a size of 25,756 bytes is hereby incorporated by reference pursuant to 37 C.F.R. § 1.834(c)(1).

### FIELD OF THE INVENTION

The disclosed invention is generally in the field of cancer treatment and specifically in the area of treatment of hepatocellular carcinoma.

### BACKGROUND OF THE INVENTION

Hepatocellular carcinoma (HCC) represents the majority form of primary liver malignancies. It is forecasted that HCC incidence will continue to rise, with around 1 million people diagnosed annually by 2025 (1). Despite the surgical advancements, HCC remains one of the leading causes of cancer death with a mortality to incidence ratio of 0.91. HCC patients are usually diagnosed at late stages when they are no longer malleable to tumor resection or local ablation. For over a decade since 2007, sorafenib, a tyrosine kinase inhibitor (TKI), has been the sole FDA-approved drug for advanced HCC patients. In the past few years, more drugs have been approved by FDA for inoperable HCC. Currently, the first-line HCC treatment options include sorafenib (TKI), lenvatinib (TKI), and combination treatment of atezolizumab (anti-PD-L1) and bevacizumab (anti-VEGF). The second-line HCC treatments for TKI-resistant tumors mainly include regorafenib (TKI), cabozantinib (TKI), ramucirumab (anti-VEGFR-2), pembrolizumab (anti-PD-1), and combination of nivolumab (anti-PD-1) and ipilimumab (anti-CTLA-4) (1). These drugs are mainly tyrosine kinase inhibitors (TKIs) with multiple targets including VEGF receptors or immune checkpoint inhibitors (ICIs) targeting T cell inhibitory receptors or their corresponding ligands. PD-1 and CTLA-4 are the most studied inhibitory receptors of T cells (1, 2). PD-1 binds with PD-L1/L2 expressed on cancer and antigen presenting cells (APCs) while CTLA-4 binds to CD80/86 expressed on APCs to inhibit T cells (3). ICIs aim to disrupt these interactions to unleash T cell activity against cancer cells (3). While clinical trials demonstrated that nivolumab or pembrolizumab had remarkable effects in a small population of HCC patients previously treated with sorafenib (4, 5), nivolumab did not show significant overall survival improvement as a first-line treatment when compared to sorafenib (6). Meanwhile, atezolizumab and bevacizumab combined treatment has more superior survival benefits in advanced HCC patients as first-line treatment than sorafenib (7). This illustrated that ICIs as a single treatment agent may not be sufficient in treating HCC and combination treatment will be the key approach in improving clinical outcomes. It is important to understand the reasons behind the unresponsiveness to ICIs and identify different targets to achieve optimal therapeutic outcomes with ICIs.

There is a need for an effective treatment for hepatocellular carcinoma (HCC).

It is therefore an objective of the present invention is to provide a method that can be used in combination with existing therapies to enhance the treatment of HCC.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### BRIEF SUMMARY OF THE INVENTION

Disclosed herein are methods for treating hepatocellular carcinoma (HCC). The methods include treating subjects with HCC that are generally unresponsive to immune checkpoint inhibitors. In some forms, the methods of treating the subjects with HCC involve administering inhibitors of the P2X7 receptor, inhibitors of nicotinamide phosphoribosyltransferase (NAMPT), or a combination thereof. In another form, the methods of treating the subjects with HCC include administering immune checkpoint inhibitors in combination with the inhibitors of the P2X7 receptor and/or inhibitors of NAMPT.

Disclosed are methods of treating a subject having a disease or condition related to elevated nicotinamide adenine dinucleotide (NAD) levels. In some forms, the subject has hepatocellular carcinoma (HCC) or has been identified as being at increased risk of developing HCC. In some forms, the methods involve administering to the subject an effective amount of an inhibitor of P2X7 receptor, an inhibitor of nicotinamide phosphoribosyltransferase (NAMPT), or a combination thereof.

In some forms, the subject has HCC. In some forms, the subject is determined to be substantially unresponsive to immune checkpoint inhibitors. In some forms, the subject is substantially unresponsive to immune checkpoint inhibitors.

In some forms of the methods, prior to the administering, the subject has elevated nicotinamide adenine dinucleotide (NAD) levels when treated with an immune checkpoint inhibitor relative to average NAD levels of HCC patients when treated with an immune checkpoint inhibitor. In some forms, the elevated NAD levels are detected in serum and/or tissues of the subject.

In some forms, the method further includes treating the subject with an immune checkpoint inhibitor at the same time or after the administering of the inhibitor of P2X7 receptor and/or the inhibitor of NAMPT.

In some forms, the administering is in combination with the immune checkpoint inhibitor. In some forms, the immune checkpoint inhibitor targets programmed cell death protein 1 (PD-1), programmed cell death ligand 1 (PD-L1), cytotoxic T-lymphocyte-associated protein 4 (CTLA-4), lymphocyte-activation gene 3 (LAG-3), or combination thereof.

In some forms, the inhibitor of P2X7 receptor is a polypeptide, a small molecule, a peptidomimetic, a nucleic acid that targets genomic or expressed nucleic acids encoding the P2X7 receptor, or a vector that encodes an inhibitory nucleic acid that targets genomic or expressed nucleic acids encoding the P2X7 receptor. In some forms, the polypeptide is an antibody. In some forms, the expressed nucleic acid is mRNA. In some forms, the inhibitor of P2X7 receptor is A438079, A-740003, AZ-10606120 or a derivative thereof.

In some forms, the inhibitor of NAMPT is a polypeptide, a small molecule, a peptidomimetic, a nucleic acid that targets genomic or expressed nucleic acids encoding NAMPT, or a vector that encodes an inhibitory nucleic acid that targets genomic or expressed nucleic acids encoding NAMPT. In some forms, the polypeptide is an antibody. In some forms, the expressed nucleic acid is mRNA. In some forms, the NAMPT inhibitor is a functional nucleic acid selected from the group consisting of an antisense molecule, siRNA, miRNA, aptamer, ribozyme, triplex forming molecule, RNAi, and external guide sequence. In some forms, the inhibitor of NAMPT is FK866, GMX1778 or a derivative thereof.

In some form, the subject is an HCC patient exhibiting elevated nicotinamide adenine dinucleotide (NAD) levels relative to HCC patients with lower NAD levels. In some forms, the elevated NAD levels in HCC patients result from the administration of immune checkpoint inhibitors. In some forms, the elevated NAD levels are detected in serum and/or tissues. In some forms, the elevated NAD levels are due to the overexpression of genes in NAD biosynthetic pathways. In some forms, the overexpression of NAD-related genes is a consequence of interferons produced by T-cells following the administration of immune checkpoint inhibitors. In some forms, the interferons upregulate the expression of genes involved in NAD biosynthetic pathways, such as genes for NAMPT, utilizing JAK/STAT signaling pathways. In some forms, NAD activates the P2X7 receptor. In some forms, the activation of the P2X7 receptor induces T cell death, T cell exhaustion, or Treg induction. In some forms, the elevation of NAD levels in serum and/or tissues indicates unresponsiveness to the administered immune checkpoint inhibitors. In some forms, serum NAD and/or tissue NAD levels in HCC can be used as a biomarker for compromised immune responses.

In some forms, the immune checkpoint inhibitor targets programmed cell death protein 1 (PD-1), programmed cell death ligand 1 (PD-L1), cytotoxic T-lymphocyte-associated protein 4 (CTLA-4), lymphocyte-activation gene 3 (LAG-3), or combination thereof.

In some forms, the inhibitor of P2X7 receptor is a small molecule, A438079, or a derivative thereof.

In some forms, the inhibitor of NAMPT can inhibit NAMPT protein and/or inhibit the genes expressing NAMPT.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIGS. 1A-1C show transcriptome analysis of 3 human HCC cell lines that revealed the upregulation of NAMPT and the enrichment of NAD biosynthesis process in HCC upon exposure to interferons. FIGS. 1D-1F show relative NAMPT mRNA expression level upon exposure to IFNγ (50 ng/mL, 24 hours) in human HCC cell lines (Hep3B, Huh7 and HepG2). FIGS. 1G-1H show Nampt mRNA expression level upon anti-PD-1 treatment in anti-PD-1 resistant HDTVi-induced mouse HCC models in C57BL/6 mice (left: Trp53^{KO}c-myc^{OE} HCCs; right: Keap1^{KO}c-myc^{OE} HCCs in mice fed with high fat dict). FIGS. 1I-1L show CIBERSORT analysis showed the correlation of NAMPT expression and estimated tumor infiltrating immune cell proportion (CD8 T cells and activated NK cells) in HCC patients from TCGA and the comparison of estimated proportion of these immune cells among HCC patients with high and low NAMPT expression (median cutoff). FIG. 1M shows histology and expression distributions of NAMPT, IFNG signature, CD8A and KLRK1 of representative anti-PD-1 responder and non-responder HCC patients from SpatialTME. *P<0.05, **P<0.01, ***<0.001, ****P<0.0001 vs. Ctrl, Vehicle or NAMPT^{low} as indicated. Student's t test.

FIGS. 2A-2F are graphs showing relative NAMPT mRNA expression levels in Hep3B-NTC, -shIFNGR1, -shIFNGR2, shJAK1, -shJAK2, -shSTAT1 and- shIRF1 upon exposure to IFNγ (50 ng/mL, 24 hours and 48 hours). FIGS. 2G-2I show in silico analysis revealed the locations of putative gamma interferon activation site element (GAS) and interferon response element (IRE; in purple), binding sites of STAT and IRF, at NAMPT promoter region. FIGS. 2H-2I show ChIP assays in Hep3B exposed to IFNγ (50 ng/mL, 24 hours) using anti-STAT1 and IgG antibodies. *P<0.05, **P<0.01, ***<0.001, ****P<0.0001 vs. NTC or IgG as indicated. Student's t test.

FIG. 3A Relative Nampt mRNA expression level of Hepa1-6-EV and-shNampt upon exposure to IFNγ (25 ng/mL, 24 hours) was determined by RT-qPCR. FIGS. 3B-3C are bar graphs showing CTV proliferation assay on (left) CD4⁺ and (right) CD8⁺ T cells co-cultured with Hepa1-6-EV and-shNampt with or without pre-treatment of IFNγ (25 ng/mL, 24 hours) for 48 hours. FIG. 3D shows relative extracellular NAD level of Hepa1-6-EV and-shNampt upon exposure to IFNγ (25 ng/mL, 48 hours) was determined LC-MS-MS. FIGS. 3E-3G shows effect of A438079 (10 μM) on cell death of CD4⁺ T cells and CD8⁺ T cells in the absence or presence of 10 μM NAD. FIGS. 3H-3L shows effect of A438079 (10 μM) on proportion of Treg cells in the absence or presence of 10 μM NAD. FIGS. 3M-3R show percentage of PD-1⁺ (top) CD4⁺ and (bottom) CD8⁺ T cells treated with indicated doses of NAD. FIGS. 3S-3U show bulk RNA sequencing performed on T cells treated with or without NAD. FIG. 3S shows expression of selected genes in T cells treated with or without NAD. FIGS. 3T-3U show GSEA analysis showed the enrichment of selected gene sets in T cells treated with NAD. *P<0.05, **P<0.01, ***<0.001, ****P<0.0001 vs. EV, —or 0 μM as indicated. Student's t test.

FIG. 4A shows schematic of subcutaneous tumors derived from Hepa1-6-EV and -shNampt that were implanted into C57L/J mice and treated with anti-PD-1 or vehicle. FIGS. 4B-4E are spider plots showing tumor growth during the experiment. FIGS. 4F-4G show tumor volumes and representative pictures of subcutaneous tumors. FIGS. 4H-4R show tumor infiltrating T cells that were analyzed by flow cytometry. FIGS. 4H-4K show quantification of CD8⁺ T cells. FIGS. 4L-4N show PD-1 and TIM-3 expressions of CD8⁺CD44⁺CD62L⁻T cells. FIG. 4O shows quantification of CD4⁺ T cells. FIGS. 4P-4R show PD-1 and TIM-3 expressions of CD4⁺CD44⁺CD62L⁻T cells. FIGS. 4S-4U show Trp53^{KO}c-myc^{OE} (EV) or Nampt^{KO}Trp53^{KO}c-myc^{OE} HCC tumors were induced by HDTVi in C57BL/6N mice and treated with anti-PD-1 or vehicle. FIGS. 4S-4T Survival plot and body weight of mice. FIG. 4U shows tumor incidence of mice. *P<0.05, **P<0.01, ***<0.001, ****P<0.0001 vs. EV-Vehicle, EV-anti-PD-1 or sh2-Vehicle as indicated. (FIGS. 4A-4R) Student's t test. (FIGS. 4S-4U) Kaplan-Meier followed by log-rank test.

FIGS. 5A-5D are CIBERSORT analysis showing the correlation of P2RX7 expression and estimated tumor infiltrating immune cell proportion (CD8 T cells and activated NK cells) in HCC patients from TCGA and the comparison of estimated proportion of these immune cells among HCC patients with high and low P2RX7 expression (median cutoff). FIGS. 5E-5F show tumor volumes and representative pictures of orthotopic tumors derived from Hepa1-6 in C57L/J mice treated with A438079 (30 mg/kg, every other day) or vehicle. FIGS. 5G-5H show representative H&E staining of HCC tumors. FIGS. 5I-5Q show quantification and representative pictures of (FIGS. 5I-5K) CD8⁺ T cells, (FIGS. 5L-5N) CD4⁺ T cells and (FIGS. 5O-5Q) NK cells in tumors. *P<0.05, ***<0.001, ****P<0.0001 vs. P2RX7^{low} or Vehicle as indicated. Student's t test. (FIGS. 5G-5H) Original, 0.77× magnification (scale bars, 2.5 mm); Inset, 20× magnification (scale bars, 100 μm). (FIGS. 51-5K) 40× magnification (scale bars, 50 μm).

FIGS. 6A-6F are plots showing Trp53^{KO}c-myc^{OE} HCC tumors that were induced by HDTVi in C57BL/6N mice and treated with different immunotherapies. FIG. 6A is a plot of generated after LC-MS-MS analysis showing the relative serum NAD levels in HDTVi-induced Trp53^{KO}c-myc^{OE} HCC bearing mice upon treatment of anti-PD-1. FIG. 6B shows survival plot and FIG. 6C shows body weight of HCC bearing mice treated with vehicle, anti-PD-1, A438079 or the combination of both. FIG. 6D is a plot of generated after LC-MS-MS analysis showing the relative serum NAD levels in HDTVi-induced Trp53^{KO}c-myc^{OE} HCC bearing mice upon treatment of CD40 agonist. FIG. 6E shows survival plot and FIG. 6F shows body weight of HCC bearing mice treated with vehicle, CD40 agonist, A438079 or the combination of both. FIGS. 6G-6H are plots showing Relative serum NAD level in HDTVi-induced Trp53^{KO}c-myc^{OE} and Keap1^{KO}c-myc^{OE} HCC bearing mice. FIG. 6I is a schematic summary of immunosuppressive role of extracellular NAD in HCC. *P<0.05, ***<0.001 vs. Vehicle or Trp53^{KO} as indicated. (FIGS. 6A-6H) Student's t test. (FIGS. 6A-6F) Kaplan-Meier followed by log-rank test.

FIG. 7 is a schematic demonstrating project summary and workflow.

FIGS. 8A-8D are two representative anti-PD-1 resistant tumor models. (FIGS. 8E-8F) Serum and (FIGS. 8G-8H) tumoral levels of NAD in immunotherapy-treated mouse HCC. (FIGS. 8I-8J) Anti-PD-1 responsive tumor model. (FIGS. 8K-8L) Trp53KO/MYCOE is an anti-PD-1 resistant mouse HCC and Keap1KO/MYCOE is an anti-PD-1 responsive mouse HCC.

### DETAILED DESCRIPTION OF THE INVENTION

The disclosed method and compositions can be understood more readily by reference to the following detailed description of particular embodiments and the Example included therein and to the Figures and their previous and following description.

### I. Definitions

Unless defined otherwise, technical and scientific terms used herein have the same meaning as commonly understood by one of ordinary skill in the art to which this disclosure pertains.

As used herein, the singular forms “a”, “an”, and “the” include both singular and plural referents unless the context clearly dictates otherwise.

“Encode,” as used in reference to a nucleotide sequence of nucleic acid encoding a gene product, e.g., a protein, of interest, is meant to include instances in which a nucleic acid contains a nucleotide sequence that is the same as the endogenous sequence, or a portion thereof, of a nucleic acid found in a cell or genome that, when transcribed and/or translated into a polypeptide, produces the gene product. In some instances, a nucleotide sequence or nucleic acid encoding a gene product does not include intronic sequences. In particular instances, a nucleotide sequence or nucleic acid encoding a T cell receptor includes a nucleotide sequence that can be translated, in silico, into an amino acid sequence corresponding to variable and constant domains of a T cell receptor, with no intervening intronic sequences.

As used herein, the term “target” or “marker” refers to any entity that is capable of specifically binding to a particular targeting moiety. In some forms, targets are specifically associated with one or more particular tissue types. In some forms, targets are specifically associated with one or more particular cell types. For example, a cell type specific marker is typically expressed at levels at least 2 fold greater in that cell type than in a reference population of cells. In some forms, the cell type specific marker is present at levels at least 3 fold, at least 4 fold, at least 5 fold, at least 6 fold, at least 7 fold, at least 8 fold, at least 9 fold, at least 10 fold, at least 50 fold, at least 100 fold, or at least 1000 fold greater than its average expression in a reference population. Detection or measurement of a cell type specific marker may make it possible to distinguish the cell type or types of interest from cells of many, most, or all other types. In some forms, a target can include a protein, a carbohydrate, a lipid, and/or a nucleic acid, as described herein. A substance is considered to be “targeted” for the purposes described herein if it specifically binds to a target. In some forms, a targeting moiety specifically binds to a target under stringent conditions. An inventive nanocarrier, such as a vaccine nanocarrier, including a targeting moiety is considered to be “targeted” if the targeting moiety specifically binds to a target, thereby delivering the entire nanocarrier to a specific organ, tissue, cell, and/or subcellular locale.

As used herein, the term “therapeutic agent” refers to any agent that, when administered to a subject, has a therapeutic, prophylactic, and/or diagnostic effect and/or elicits a desired biological and/or pharmacological effect.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64,65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “monitoring” as used herein refers to any method in the art by which an activity can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the compounds of the invention.

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

The terms “individual”, “host”, “subject”, and “patient” are used interchangeably, and refer to a mammal, including, but not limited to, murids, simians, humans, mammalian farm animals and livestock, mammalian sport animals, and mammalian pets.

“Treatment” or “treating” means to administer a composition to a subject or a system with an undesired condition (e.g., HCC). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition. The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

The term “dosage regime” refers to drug administration regarding formulation, route of administration, drug dose, dosing interval and treatment duration.

The term “pharmaceutically acceptable” or “biocompatible” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be acceptable” in the sense of being compatible with the other ingredients of a subject composition and not injurious to the patient.

The terms “inhibit” or “reduce” in the context of inhibition, mean to reduce, or decrease in activity and quantity. This can be a complete inhibition or reduction in activity or quantity, or a partial inhibition or reduction. Inhibition or reduction can be compared to a control or to a standard level. Inhibition can be measured as a % value, e.g., from 1% up to 100%, such as 5%, 10, 25, 50, 75, 80, 85, 90, 95, 99, or 100%. For example, compositions including a disclosed inhibitor may inhibit or reduce the activity and/or quantity of one or more disclosed mechanisms, pathways, or symptoms by about 10%, 20%, 30%, 40%, 50%, 75%, 85%, 90%, 95%, or 99% from the activity and/or quantity of the same inhibitor in subjects that did not receive or were not treated with the compositions. In some forms, the inhibition and reduction are compared according to the level of mRNAs, proteins, cells, tissues, and organs.

The terms “immunologic”, “immunological” or “immune” response refer to the development of a beneficial humoral (antibody mediated) and/or a cellular (mediated by antigen-specific T cells or their secretion products) response directed against an immunogen in a recipient patient. Such a response can be an active response induced by administration of immunogen or a passive response induced by administration of antibody or primed T-cells. The relative contributions of humoral and cellular responses to the protective or therapeutic effect of an immunogen can be distinguished by separately isolating antibodies and T-cells from an immunized syngeneic animal and measuring protective or therapeutic effect in a second subject.

As used herein, the term “immunostimulatory agent” refers to an agent that modulates an immune response to an antigen but is not the antigen or derived from the antigen. “Modulate”, as used herein, refers to inducing, enhancing, suppressing, directing, or redirecting an immune response. Such agents include immunostimulatory agents that stimulate (or boost) an immune response to an antigen but as defined above, is not the antigen or derived from the antigen. Immunostimulatory agents, therefore, include adjuvants. In some forms, the immunostimulatory agent is on the surface of the nanocarrier and/or is encapsulated within the nanocarrier. In some forms, the immunostimulatory agent on the surface of the nanocarrier is different from the immunostimulatory agent encapsulated within the nanocarrier. In some forms, the nanocarrier includes more than one type of immunostimulatory agent. In some forms, the more than one type of immunostimulatory agent act on different pathways. Examples of immunostimulatory agents include those provided elsewhere herein.

In general, a “small molecule” is understood in the art to be an organic molecule that is less than about 2000 g/mol in size. In some forms, the small molecule is less than about 1500 g/mol or less than about 1000 g/mol. In some forms, the small molecule is less than about 800 g/mol or less than about 500 g/mol. In some forms, small molecules are non-polymeric and/or non-oligomeric. In some forms, small molecules are not proteins, peptides, or amino acids. In some forms, small molecules are not nucleic acids or nucleotides. In some forms, small molecules are not saccharides or polysaccharides.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. Methods for Detecting Immunotherapy Resistance in HCC Treatment

Current treatment strategies for hepatocellular carcinoma (HCC) rely primarily on systemic therapies including tyrosine kinase inhibitors (TKIs) and immune checkpoint inhibitors (ICIs). While ICIs targeting PD-1/PD-L1 or CTLA-4 have shown some clinical benefit, their efficacy remains limited to a subset of patients. The biological mechanisms underlying ICI resistance in HCC remain poorly defined, and currently available biomarkers, such as tumor mutational burden (TMB), PD-L1 expression, and CD8⁺ T cell infiltration, have demonstrated inconsistent predictive value. These markers are highly variable across tumor regions, often require invasive biopsies, and may not reflect the dynamic immune interactions occurring in the tumor microenvironment. As a result, clinicians lack reliable tools to predict which HCC patients will respond to ICI therapy, and many patients experience treatment failure or disease progression despite receiving immunotherapy.

HCC exhibits a complex immunosuppressive microenvironment that evolves in response to treatment. Recent studies have identified limitations in the clinical efficacy of immune checkpoint inhibitors (ICIs) in HCC, with only a subset of patients exhibiting durable responses. The mechanisms underlying this resistance are not fully understood, but emerging evidence suggests that treatment-induced interferon-gamma (IFN-γ) signaling may contribute to immunosuppression within the tumor microenvironment. IFN-γ has been shown to induce the expression of immunoregulatory genes, such as PD-L1 and indoleamine-2,3-dioxygenase (IDO), which suppress effector T cell activity and facilitate immune evasion. However, these known pathways do not fully account for the extent of ICI resistance observed in HCC, indicating the involvement of additional, yet unidentified, mechanisms.

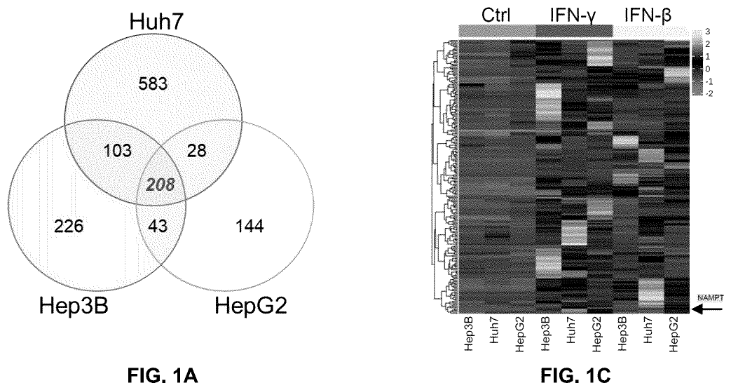

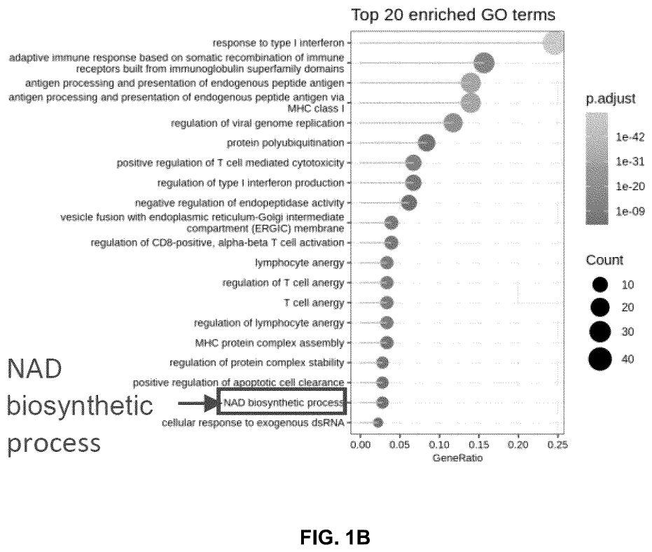

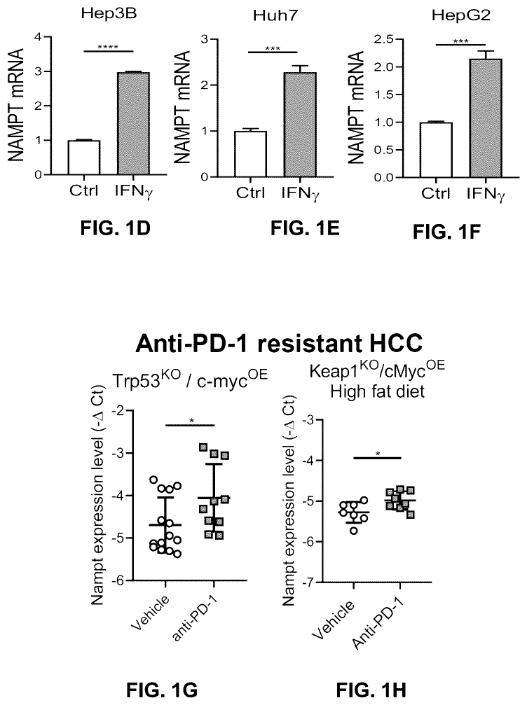

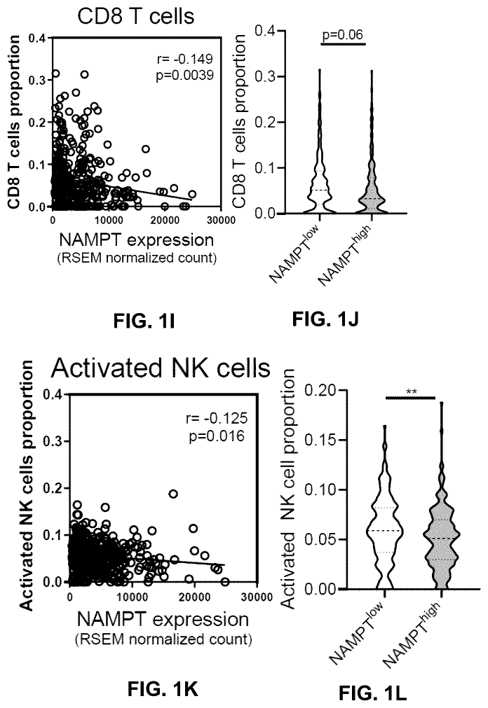

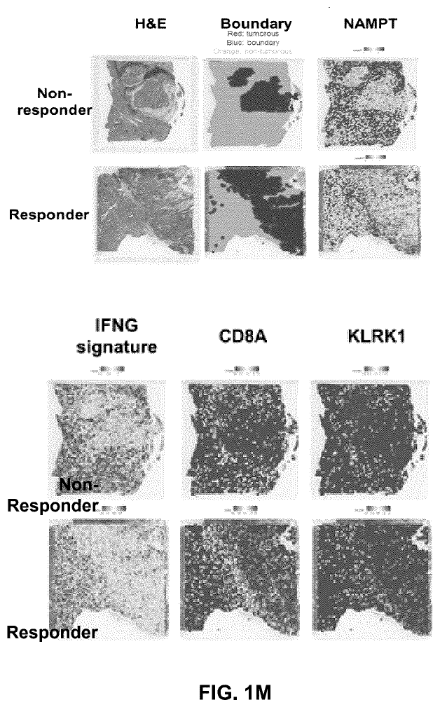

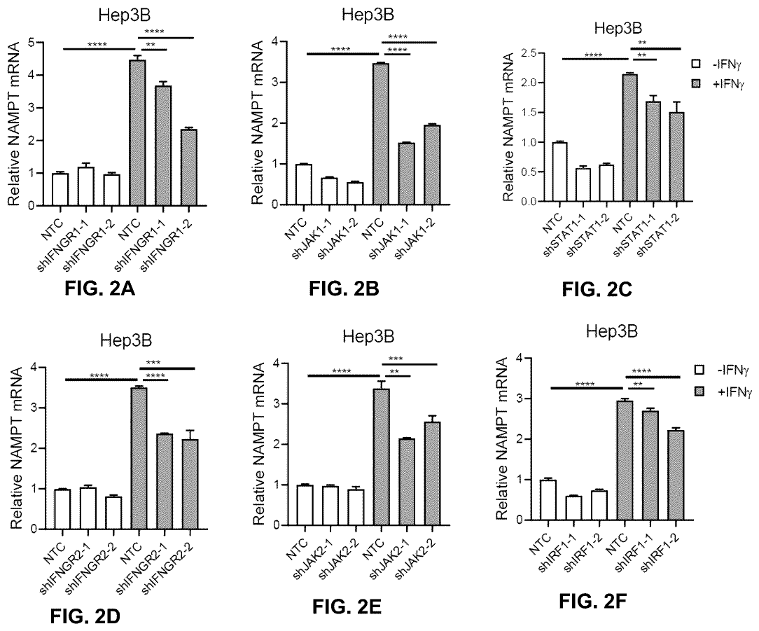

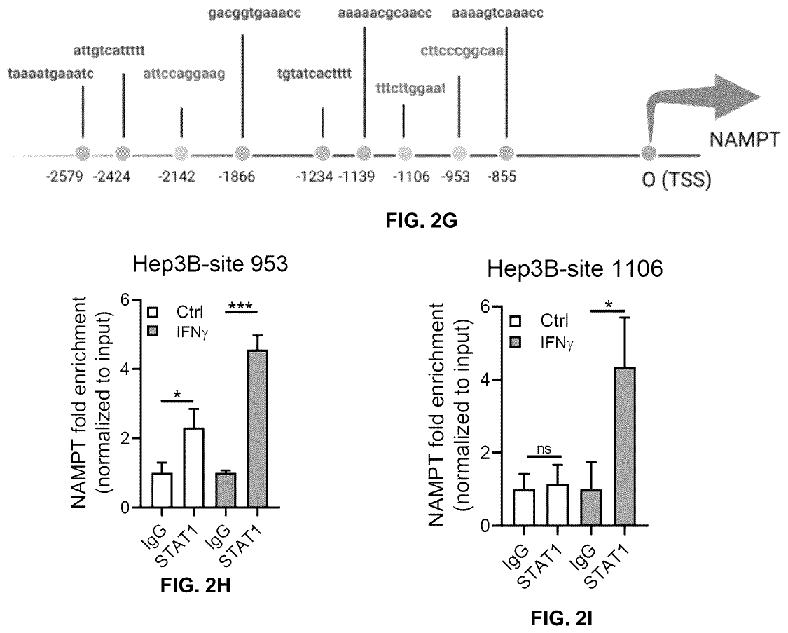

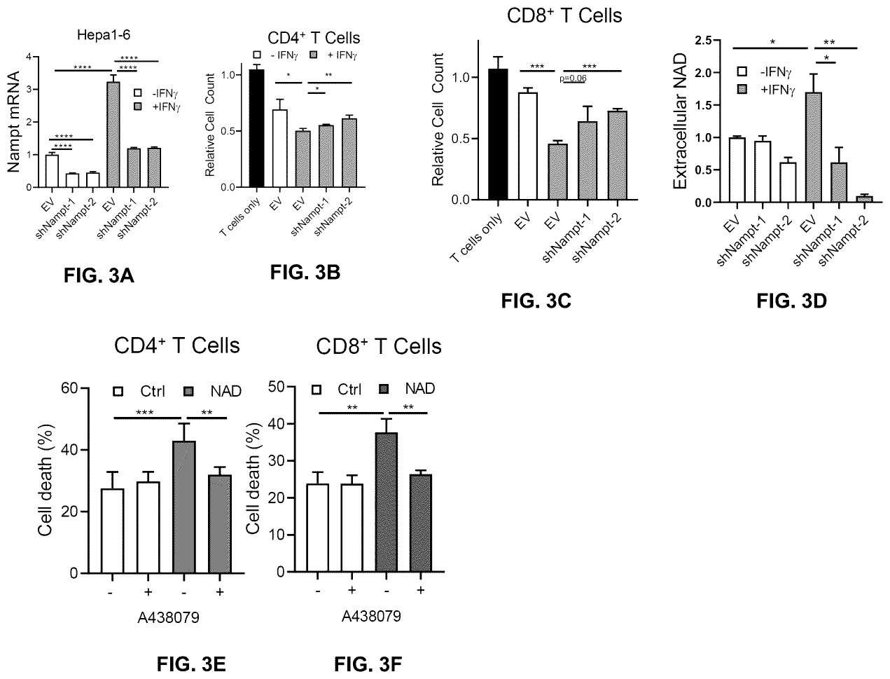

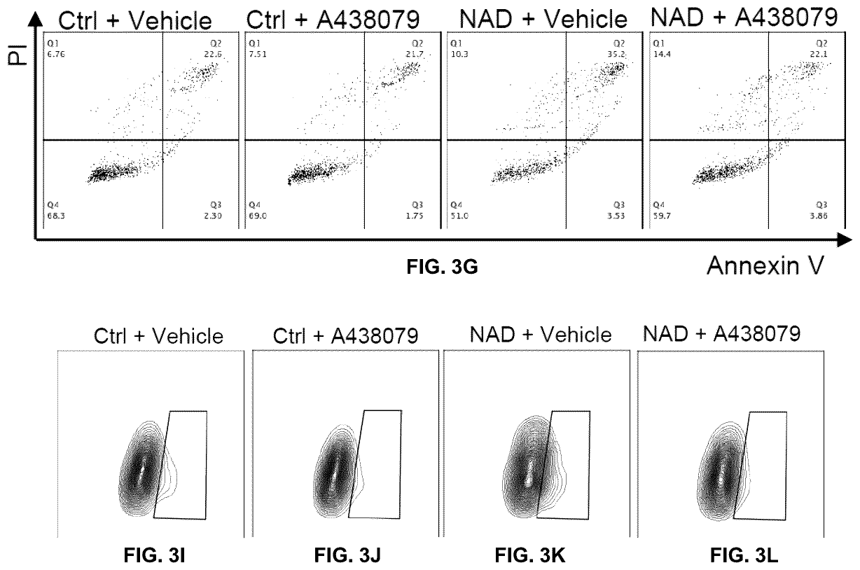

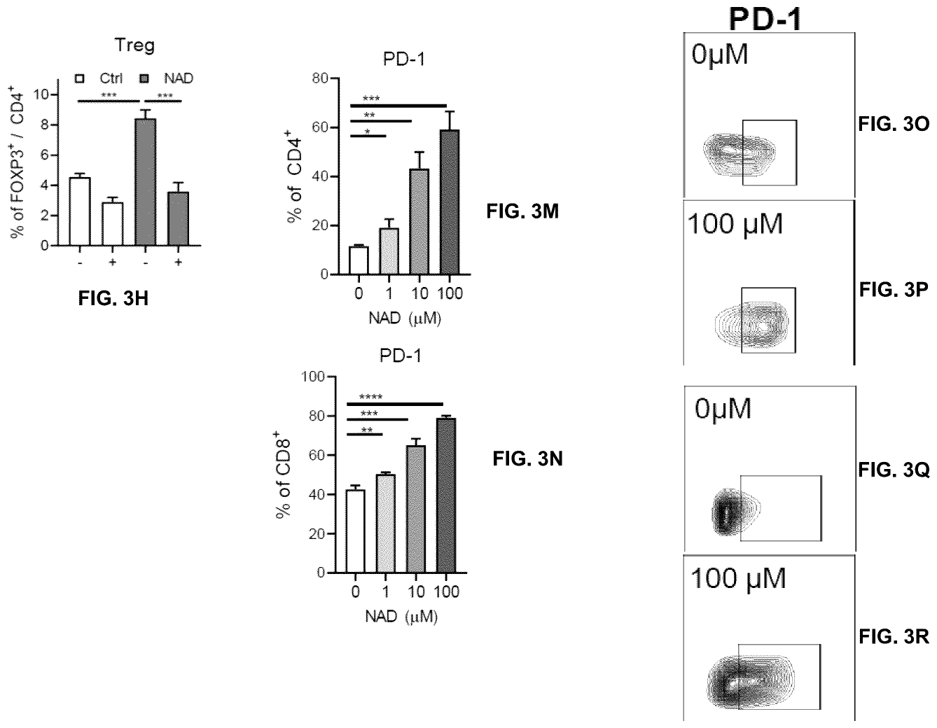

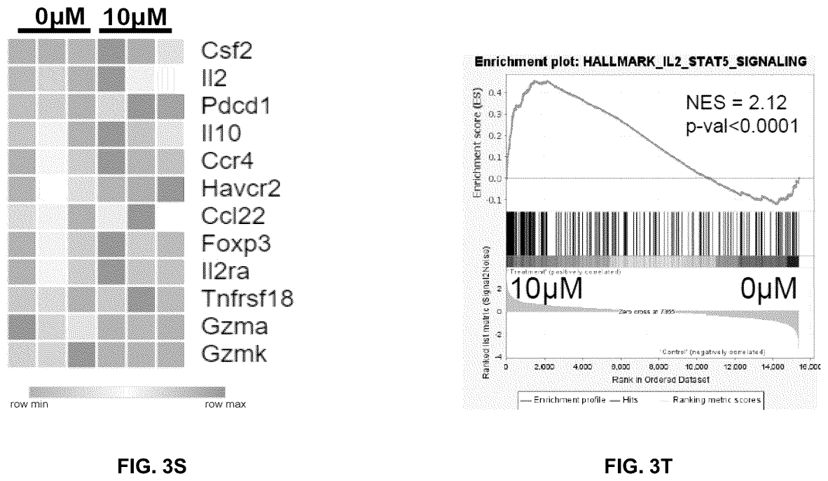

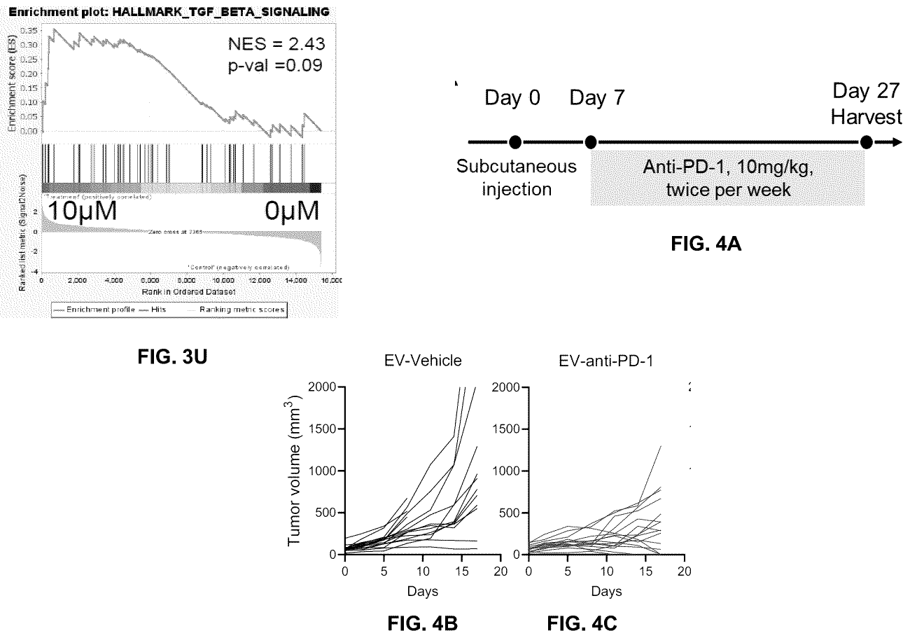

To address these challenges, methods for identifying or stratifying patients who are less likely to respond to ICI monotherapy based on circulating NAD levels are provided. The disclosed methods are based on the discovery that NAD metabolism plays an important role in modulating the immune response to ICIs in HCC (see Examples, FIGS. 1A-1E, 2A and 2B). The non-limiting examples demonstrate that the rate-limiting NAD biosynthetic enzyme NAMPT is upregulated in response to IFN-γ, leading to increased extracellular NAD levels (see Examples, FIGS. 1A-1E, 2A and 2B). It was also demonstrated that extracellular NAD suppresses anti-tumor immunity by promoting T cell apoptosis, inducing T cell exhaustion, and increasing regulatory T cell (Treg) differentiation via the P2X7 receptor (see Examples, FIGS. 3A-3D). These findings support the use of serum NAD levels as a potential biomarker for predicting immunotherapy response in HCC patients.

Provided are methods for detecting the level of NAD in liquid biopsies (e.g., blood or serum samples) for predicting response to ICI therapy in HCC. These biomarker-based methods offer an alternative to conventional approaches such as tumor biopsies and commonly used biomarkers like PD-L1 expression or TMB, which are often invasive, limited by tumor heterogeneity, or impractical for repeated monitoring. For example, the disclosed methods provide increased specificity for identifying patients likely to exhibit resistance to ICI therapy, compared to existing biomarkers such as tumor mutational burden, PD-L1 expression, or CD8⁺ T cell infiltration, which show variable predictive performance across studies and often require tissue samples.

In the context of HCC, NAD serves as a soluble metabolic biomarker that reflects underlying tumor-driven immunosuppressive mechanisms associated with poor response to immunotherapy. NAD levels are elevated in patients or animal models harboring ICI-resistant tumors and are induced by IFN-γ signaling following ICI exposure. This elevation is driven by upregulation of the NAD biosynthetic enzyme NAMPT and leads to accumulation of extracellular NAD, which promotes T cell apoptosis, immune exhaustion, and regulatory T cell (Treg) differentiation through activation of the P2X7 receptor. As such, serum NAD can be used to indicate a tumor's immunosuppressive status and resistance potential. The method detects these changes by measuring NAD levels in a subject-derived sample (e.g., blood or serum), thereby providing predictive information to guide selection of therapeutic strategy, including the use of NAMPT or P2X7R inhibitors in combination with ICI therapy. Methods of detecting and measuring NAD levels in biological samples are further described below.

### 1. Sources of Samples

The NAD biomarker described above can be detected in a variety of biological samples obtained from a subject. In preferred embodiments, the biological sample is blood, such as plasma, serum, or whole blood, which is suitable for analysis using rapid detection platforms or clinical laboratory assays.

The NAD biomarker can be incorporated into diagnostic devices such as lateral flow assays or biosensors for evaluating immunotherapy response in HCC. For example, the diagnostic device can be a lateral flow assay containing a test strip with immobilized capture agents (e.g., enzymes or affinity reagents) specific for NAD+ or NAD+ metabolites such as quinolinic acid. An exemplary lateral flow assay can also include a detection reagent coupled to a colorimetric or fluorescent label to permit visual or instrumental readout of NAD+ concentration in the blood sample. In other forms, the diagnostic device can be a biosensor containing a functionalized sensor surface capable of binding NAD or NAD metabolites (e.g., quinolinic acid) in real time, thereby allowing continuous profiling of circulating NAD levels during or prior to ICI treatment.

While blood and blood components (e.g., serum or plasma) are preferred for their accessibility and suitability for longitudinal sampling, NAD can also be detected in other biological fluids such as urine, saliva, or interstitial fluid, depending on the detection platform and clinical setting. In some forms, NAD can be detected in its oxidized or reduced forms, or in conjunction with associated biosynthetic enzymes (e.g., NAMPT) as indicators of metabolic state or immune suppression.

Alternatively, NAD or related metabolites (e.g., quinolinic acid) can be purified or isolated from the biological sample using known extraction techniques, such as enzymatic cycling assays, colorimetric kits, or mass spectrometry-based protocols. In these forms, the NAD detected can originate from tumor cells, stromal cells, or infiltrating immune cells within the tumor microenvironment, and may be released into circulation through active transport, cell lysis, or exosomal secretion. In some forms, associated substrates such as NAMPT protein or mRNA can be extracted from cells present in blood (e.g., circulating tumor cells, monocytes, or lymphocytes) or from cell-free components such as serum or plasma.

### 2. Sample Collection and Molecular Analysis

The disclosed methods for detecting NAD+ and/or its metaboliztes utilize blood or serum as the primary source of the biological sample. Blood samples can be collected using standard clinical methods, such as venipuncture into collection tubes containing anticoagulants (e.g., EDTA, heparin, or citrate) to prevent clotting and preserve sample integrity. Plasma may be isolated by centrifuging whole blood at 2,000-3,500×g for 10-15 minutes at 4° C., while serum can be prepared by allowing blood to clot at room temperature followed by centrifugation. To preserve NAD and related proteins or enzymes, stabilizing agents such as protease inhibitors (e.g., PMSF, aprotinin) or protein stabilizers (e.g., glycerol, BSA) can be added post-collection. Samples may be stored at −80° C. to prevent degradation prior to analysis.

Generally, the level of NAD is determined by quantifying oxidized or reduced forms, or associated metabolites, using analytical platforms such as enzymatic cycling assays, colorimetric or fluorometric detection, biosensors, or mass spectrometry. In certain embodiments, sample processing may include depletion of high-abundance plasma proteins (e.g., albumin, immunoglobulins) using commercially available kits or columns, to improve sensitivity in detecting low-abundance biomarkers such as extracellular NAD or NAMPT. Protein concentration can be assessed using standard methods (e.g., BCA or Bradford assay), and enzymatic digestion (e.g., trypsin) may be performed to prepare peptides for mass spectrometry if desired.

Although analysis of NAD alone is contemplated, in some forms, the methods further include measuring one or more related metabolic or enzymatic markers, such as NAMPT or other components of the NAD biosynthetic pathway, to increase diagnostic accuracy for resistance to immunotherapy.

NAD and related biomarkers can be detected using a variety of molecular techniques, including enzyme-linked immunosorbent assays (ELISA), lateral flow assays (LFA), sandwich immunoassays, immunofluorescent assays, radioimmunoassays, chemiluminescence-based tests, fluorescence polarization immunoassays (FPIA), biosensors, and rapid point-of-care diagnostic devices. More advanced formats may employ microfluidic systems, MEMS technologies, lab-on-a-chip platforms, or nanotechnology-based biosensors. In some forms, continuous monitoring devices are employed, incorporating sensor surfaces functionalized with NAD-specific capture agents for real-time measurement. These methods can be used independently or in combination with nucleic acid-based approaches (e.g., RT-qPCR or NGS) targeting NAMPT or related transcripts, enabling integrated proteomic and transcriptomic profiling of immunometabolic signatures in HCC.

In some forms, NAD or NAMPT may be detected in the cell-free fraction (e.g., plasma or serum) or from immune cells (e.g., monocytes or lymphocytes) present in the blood. The detected NAD may reflect immunosuppressive processes in the tumor microenvironment, such as T cell exhaustion or Treg induction, which are associated with poor response to ICIs.

### 3. Diagnosis of Resistance to HCC Immunotherapy

The disclosed methods include measuring the level of NAD, and in some forms, one or more related biomarkers such as NAMPT or NAD-associated metabolites, to aid in the diagnosis, prediction, and monitoring of resistance to ICI therapy in HCC. NAD serves as a functional biomarker that reflects the immunosuppressive state of the tumor microenvironment, and elevated levels of NAD have been associated with reduced responsiveness to ICIs. Measuring NAD levels in a blood, plasma, or serum sample provides clinically actionable information regarding a subject's likelihood of responding to ICI therapy, and stratifying patients for monotherapy versus combination regimens. This approach facilitates early identification of immunotherapy-resistant tumors and supports the implementation of more effective, personalized treatment strategies. Based on the results, additional therapeutic interventions, such as co-administration of NAMPT or P2X7R inhibitors, may be recommended to overcome resistance and improve treatment efficacy.

The disclosed methods include a first step of detecting the concentration of NAD in a biological sample obtained from a subject, such as a blood, plasma, or serum sample, using one or more detection platforms (e.g., enzymatic cycling assays, colorimetric assays, ELISA, biosensors, or mass spectrometry). In some forms, the method may optionally include detection of NAMPT expression or NAD pathway-related metabolites to complement NAD measurement. In a second step, the measured NAD level is compared to a diagnostic threshold or reference value, which distinguishes between an ICI-responsive versus ICI-resistant tumor state. A serum NAD concentration above the threshold is indicative of an immunosuppressive tumor microenvironment, associated with T cell exhaustion, reduced CD8⁺ T cell infiltration, or increased Treg differentiation, all of which are linked to diminished ICI efficacy. These thresholds are established based on observed differences in NAD levels between ICI-responsive and ICI-resistant HCC models and patient populations. NAD detection can also be performed longitudinally to monitor dynamic changes during treatment, assess resistance onset, or evaluate response to adjunct therapies. In some forms, NAD quantification can be integrated into clinical decision-making workflows to guide therapeutic planning, triage patients to combination immunotherapy protocols, or identify candidates for targeted interventions aimed at restoring anti-tumor immunity.

### III. Methods of Treatment

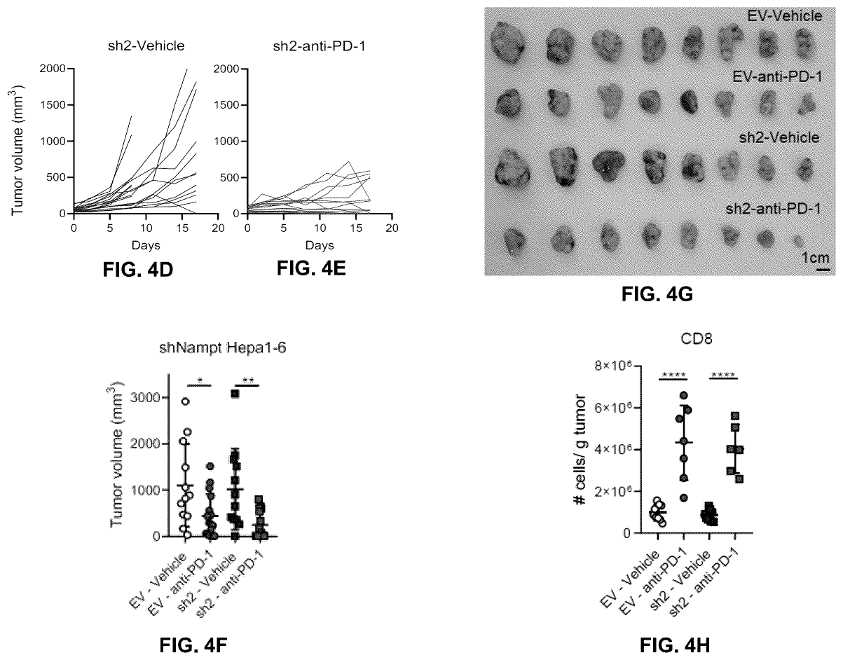

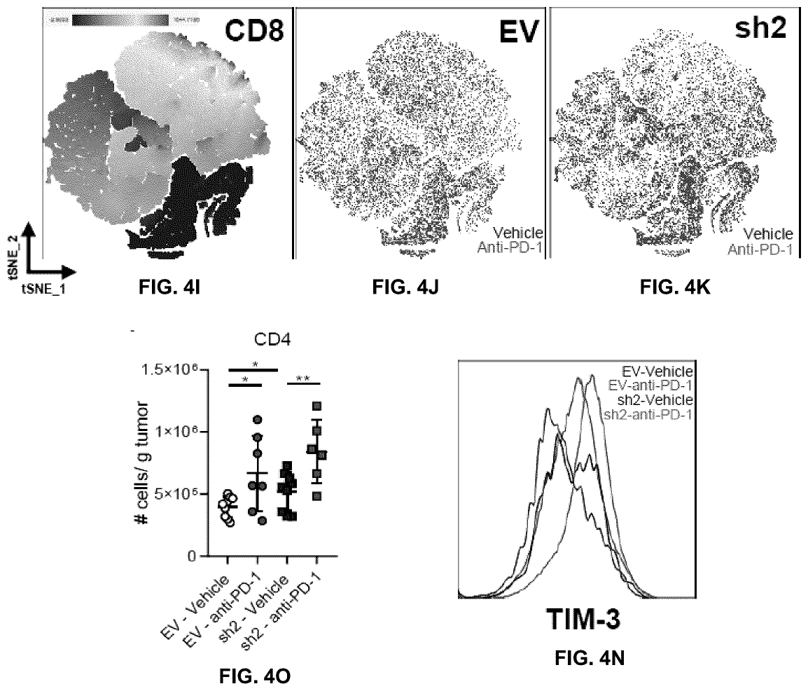

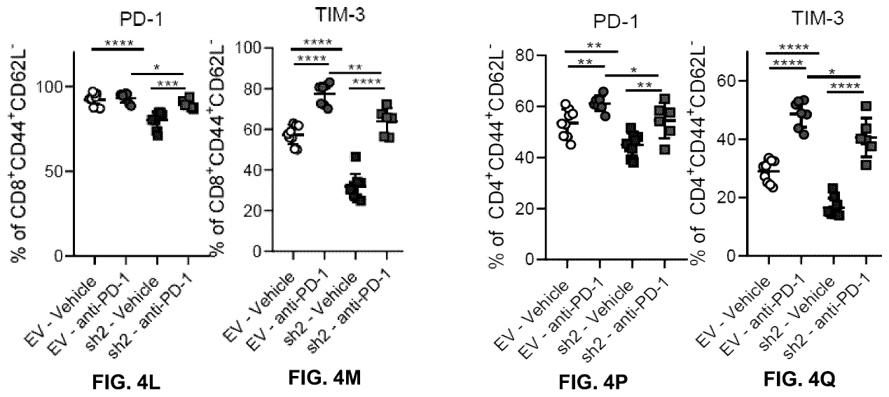

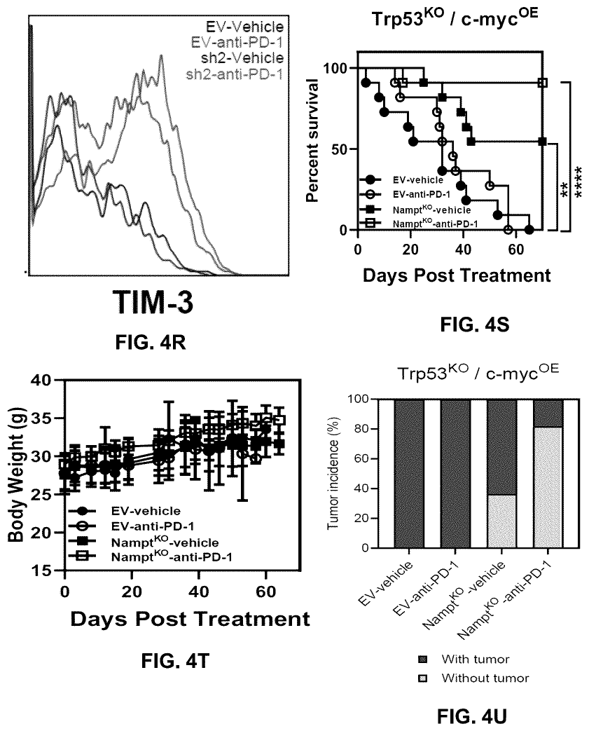

Also provided are methods and compositions for overcoming ICI resistance in HCC by targeting the NAD-NAMPT-P2X7R immunosuppressive axis. In one exemplary form, the methods include the administration of NAMPT inhibitors and/or P2X7 receptor antagonists to reduce extracellular NAD levels, thereby restoring effector T cell function, reducing regulatory T cell (Treg) accumulation, and increasing anti-tumor immune responses. In another exemplary form, the methods include the co-administration of NAMPT or P2X7R inhibitors with ICIs, such as anti-PD-1 or anti-PD-L1 antibodies, to reduce cell proliferation and size of HCC tumors resistant to immunotherapy. These methods of treatment are based on the findings that elevated serum NAD is associated with poor CD8⁺ T cell infiltration and resistance to anti-PD-1 treatment in preclinical HCC models. Further demonstrated is that pharmacological inhibition of NAMPT or blockade of the P2X7 receptor restores T cell function and increases the therapeutic efficacy of ICIs (see Examples, FIGS. 4B-4E).

### 1. Diseases to Be Treated

Methods of treating diseases and/or disorders in a subject in need thereof are provided. The subject to be treated can have a disease, disorder, or condition such as but not limited to, cancer, an immune system disorder such autoimmune disease, an inflammatory disease, a neuronal disorder, HIV/AIDS, diabetes, a cardiovascular disease, an infectious disease, or combinations thereof.

i. Cancer

In some forms, the methods treat or prevent cancer. In some forms, the methods treat or prevent cancer or other proliferative disease or disorder in a subject identified as having, or at risk of having cancer or other proliferative disease or disorder. Cancer is a disease of genetic instability, allowing a cancer cell to acquire the hallmarks proposed by Hanahan and Weinberg, including (i) self-sufficiency in growth signals; (ii) insensitivity to anti-growth signals; (iii) evading apoptosis; (iv) sustained angiogenesis; (v) tissue invasion and metastasis; (vi) limitless replicative potential; (vii) reprogramming of energy metabolism; and (viii) evading immune destruction (Cell., 144:646-674, (2011)).

Tumors, which can be treated in accordance with the disclosed methods, are classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

TABLE 4
The disclosed compositions and methods can be
used in the treatment of one or more cancers.

| | | | | |
|---|---|---|---|---|
| Acute | Acute | Adrenocortical | AIDS-Related | Kaposi |
| Lymphoblastic | Myeloid | Carcinoma | Cancers | Sarcoma |
| Leukemia | Leukemia | | | |
| (ALL) | (AML) | | | |
| AIDS-Related | Primary CNS | Anal Cancer | Appendix | Astrocytomas |
| Lymphoma | Lymphoma | | Cancer | |
| | | | (Gastrointestinal | |
| | | | Carcinoid | |
| | | | Tumors) | |
| Atypical | Brain Cancer | Basal Cell | Bile Duct | Bladder |
| Teratoid/ | | Carcinoma of the | Cancer | Cancer |
| Rhabdoid | | Skin | | |
| Tumor | | | | |
| Bone Cancer | Brain Tumors | Breast Cancer | Bronchial | Burkitt |
| (includes | | | Tumors | Lymphoma |
| Ewing | | | | |
| Sarcoma and | | | | |
| Osteosarcoma | | | | |
| and Malignant | | | | |
| Fibrous | | | | |
| Histiocytoma) | | | | |
| Non-Hodgkin | Carcinoid | Carcinoma of | Cardiac | Embryonal |
| Lymphoma | Tumors | Unknown Primary | (Heart) | Tumors |
| | | | Tumors | |
| Germ Cell | Primary CNS | Cervical Cancer | Cholangio- | Chordoma |
| Tumor | Lymphoma | | carcinoma | |
| Chronic | Chronic | Chronic | Colorectal | Cranio- |
| Lymphocytic | Myelogenous | Myeloproliferative | Cancer | pharyngioma |
| Leukemia | Leukemia | Neoplasms | | |
| (CLL) | (CML) | | | |
| Cutaneous T- | Ductal | Endometrial | Ependymoma | Esophageal |
| Cell | Carcinoma In | Cancer | | Cancer |
| Lymphoma | Situ (DCIS) | | | |
| (Mycosis | | | | |
| Fungoides and | | | | |
| Sézary | | | | |
| Syndrome) | | | | |
| Esthesioneuro- | Ewing | Extracranial Germ | Eye Cancer | Intraocular |
| blastoma | Sarcoma | Cell Tumor | | Melanoma |
| Fallopian | Fibrous | Osteosarcoma | Gallbladder | Gastric Cancer |
| Tube Cancer | Histiocytoma | | Cancer | |
| | of Bone | | | |
| Stomach | Gastrointestinal | Gastrointestinal | Central | Extracranial |
| Cancer | Carcinoid | Stromal Tumors | Nervous | Germ Cell |
| | Tumor | (GIST) | System Germ | Tumors |
| | | | Cell Tumors | |
| Extragonadal | Ovarian Germ | Testicular Cancer | Gestational | Hairy Cell |
| Germ Cell | Cell Tumors | | Trophoblastic | Leukemia |
| Tumors | | | Disease | |
| Head and | Heart Tumors | Hepatocellular | Histiocytosis | Hodgkin |
| Neck Cancer | | (Liver) Cancer | (Langerhans | Lymphoma |
| | | | Cell) | |
| Hypopharyngeal | Intraocular | Islet Cell Tumors | Pancreatic | Kidney Cancer |
| Cancer | Melanoma | | Neuroendocrine | |
| | | | Tumors | |
| Renal Cell | Langerhans | Laryngeal Cancer | Leukemia | Lip and Oral |
| Cancer | Cell | | | Cavity Cancer |
| | Histiocytosis | | | |
| Liver Cancer | Lung Cancer | Lymphoma | Male Breast | Malignant |
| | (Non-Small | | Cancer | Fibrous |
| | Cell and Small | | | Histiocytoma |
| | Cell) | | | of Bone and |
| | | | | Osteosarcoma |
| Melanoma | Intraocular | Merkel Cell | Malignant | Metastatic |
| | (Eye) | Carcinoma (Skin | Mesothelioma | Cancer |
| | Melanoma | Cancer) | | |
| Metastatic | Midline Tract | Mouth Cancer | Multiple | Multiple |
| Squamous | Carcinoma | | Endocrine | Myeloma/Plasma |
| Neck Cancer | With NUT | | Neoplasia | Cell |
| with Occult | Gene Changes | | Syndromes | Neoplasms |
| Primary | | | | |
| Mycosis | Myelodysplastic | Myelodysplastic/ | Nasal Cavity | Nasopharyngeal |
| Fungoides | Syndromes | Myeloproliferative | and Paranasal | Cancer |
| (Lymphoma) | | Neoplasms | Sinus Cancer | |
| Neuroblastoma | Non-Small | Oral Cancer | Oropharyngeal | Ovarian |
| | Cell Lung | | Cancer | Cancer |
| | Cancer | | | |
| Pancreatic | Papillomatosis | Paraganglioma | Paranasal | Parathyroid |
| Cancer | | | Sinus and | Cancer |
| | | | Nasal Cavity | |
| | | | Cancer | |
| Penile Cancer | Pharyngeal | Pheochromocytoma | Pituitary | Plasma Cell |
| | Cancer | | Tumor | Neoplasm/ |
| | | | | Multiple |
| | | | | Myeloma |
| Pleuropulmonary | Primary | Primary Peritoneal | Prostate | Rectal Cancer |
| Blastoma | Central | Cancer | Cancer | |
| | Nervous | | | |
| | System (CNS) | | | |
| | Lymphoma | | | |
| Recurrent | Retinoblastoma | Rhabdomyosarcoma | Salivary | Sarcoma |
| Cancer | | | Gland Cancer | |
| Vascular | Uterine | Sézary Syndrome | Small Cell | Small Intestine |
| Tumors | Sarcoma | (Lymphoma) | Lung Cancer | Cancer |
| Soft Tissue | Squamous | Stomach (Gastric) | Throat Cancer | Thymoma |
| Sarcoma | Cell | Cancer | | |
| | Carcinoma | | | |
| Thymic | Thyroid | Transitional Cell | Carcinoma of | Ureter and |
| Carcinoma | Cancer | Cancer of the | Unknown | Renal Pelvis |
| | | Renal Pelvis and | Primary | |
| | | Ureter | | |
| Transitional | Urethral | Uterine Cancer | Vaginal | Vulvar Cancer |
| Cell Cancer | Cancer | | Cancer | |
| Wilms Tumor | | | | |

The disclosed compositions and methods of treatment thereof are generally suited for treatment of carcinomas, sarcomas, lymphomas and leukemias. The described compositions and methods are useful for treating, or alleviating subjects having benign or malignant tumors by delaying or inhibiting the growth/proliferation or viability of tumor cells in a subject, reducing the number, growth or size of tumors, inhibiting or reducing metastasis of the tumor, and/or inhibiting or reducing symptoms associated with tumor development or growth.

The types of cancer that can be treated with the provided compositions and methods include, but are not limited to, cancers such as vascular cancer such as multiple myeloma, adenocarcinomas and sarcomas, of bone, bladder, brain, breast, cervical, colorectal, esophageal, kidney, liver, lung, nasopharangeal, pancreatic, prostate, skin, stomach, and uterine. The experiments below support the conclusion that the disclosure approach is effective for treating solid tumors. Thus, in some forms, the target cancer is a solid tumor. In some forms, the compositions are used to treat multiple cancer types concurrently. The compositions can also be used to treat metastases or tumors at multiple locations.

Exemplary tumor cells include, but are not limited to, tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as, but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytom and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (cpidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, and carcinosarcoma. For a review of such disorders, sec Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

### 2. Identifying Subjects for Treatment

Any of the described methods can include one or more steps of identifying a subject to be treated for any of the conditions mentioned herein, including, but not limited to hepatocellular carcinoma (HCC).

In some forms, the subject to be treated has HCC or is at a risk of getting HCC.

In some form, the subject to be treated is substantially unresponsive to immune checkpoint inhibitors.

In certain forms, the subject to be treated are HCC patients exhibiting elevated nicotinamide adenine dinucleotide (NAD) levels relative to HCC patients with lower NAD levels. In some forms, the elevated NAD levels in HCC patients result from the administration of immune checkpoint inhibitors. In some forms, the elevated NAD levels can be found in serum and/or tissues.

Nicotinamide adenine dinucleotide (NAD) plays a role in many important biological events. Intracellular NAD is a co-factor for enzymes involving in multiple metabolic pathways including glycolysis, oxidative decarboxylation, fatty acid β-oxidation and TCA cycle (23). NAD is also needed for non-metabolic enzymes such as Sirtuins (SIRTs), ADP-ribose synthases (CD38 and CD157) and poly (ADP-ribose) polymerases (PARPs) (23). In cancers, given the key role of NAD in these biological pathways, NAD supports cancer cell energy metabolism, proliferation, survival and invasion (24, 25). Moreover, intracellular NAD is also involved in mediating tumor immune escape. Intracellular NAD drove PD-L1 transcription in HCC. Particularly, NAD supported the production of a-ketoglutarate, a co-substrate for the DNA demethylase, TET1. TET1 promoted PD-L1 transcription via STAT1/IRF1 (26). NAD could also be transported extracellularly by transporter, CX43 (27) and extracellular NAD suppressed T cells. It has been shown that extracellular NAD activated P2X7 receptor (P2X7R) and induced T cell apoptosis in an ADP-ribosyltransferase (ART) dependent manner (28). In a non-small cell lung cancer (NSCLC) model, tumor-infiltrating CD8⁺ T cells were susceptible to NAD-induced cell death mediated by ART1 (29). It was showed that P2X7R deficient tumor infiltrating CD8⁺ T cells demonstrated enhanced cytotoxic functions and better suppressed tumor growth (30).

NAD is produced in cells through three major pathways (31). The first one is the de novo synthesis pathway. In this pathway, tryptophan is first metabolized to quinolinic acid (QA) via multiple steps. QA is then converted to nicotinic acid mononucleotide (NAMN) by quinolinate phosphoribosyltransferase (QPRT). NAD is produced from NAMN via nicotinamide nucleotide adenylyltransferase (NMNAT) and NAD synthetase (NADS). The second one is the Preiss-Handler pathway in which nicotinic acid (NA) is converted to NAMN by nicotinate phosphoribosyltransferase (NAPRT), and then further converted to NAD through NMNAT and NADS. The third one is the salvage pathway. The rate-limiting enzyme, nicotinamide phosphoribosyltransferase (NAMPT), generates nicotinamide mononucleotide (NMN) from nicotinamide (NAM). NMN is further converted into NAD by NMNAT. The salvage pathway is considered a key one in NAD metabolism as it directly recycles NAM, which is produced by the enzymes using NAD as a co-factor. NAMPT has been found to be overexpressed in many cancer types and inhibition of NAMPT altered cancer cell metabolism via limiting NAD availability and suppressed tumor growth (32, 33).

Different methods that can be used to detect NAD levels in subjects with HCC. These methods include but are not limited to:
Enzymatic Cycling Assays: The method utilizes specific enzymes to catalyze the cycling of NAD/NADH, generating a measurable product (colorimetric or fluorometric signal). This is suitable for high-throughput screening and can be performed on blood, plasma, or tissue samples. Some examples include commercially available NAD/NADH quantification kits.
High-Performance Liquid Chromatography (HPLC): HPLC separates NAD and NADH based on their different polarities using chromatographic techniques, followed by UV or fluorescence detection. This can be used for precise quantification of NAD levels in plasma, serum, or tissue extracts.
Liquid Chromatography-Tandem Mass Spectrometry (LC-MS/MS): LC-MS/MS combines liquid chromatography for separation with mass spectrometry for detection and quantification of NAD and its metabolites. This technique is highly sensitive and specific and can be used for complex biological samples such as blood, urine, or tissue extracts.
Capillary Electrophoresis: This technique separates NAD and NADH based on their charge and size using an electric field, followed by detection. This can be effective for analyzing small volumes of patient samples, such as blood or cell extracts. An example includes capillary electrophoresis with laser-induced fluorescence detection.
Fluorescence-Based Assays: These kinds of assays use fluorescent dyes or probes that specifically bind to NAD or NADH, producing a fluorescence signal that can be quantified. This can be useful for real-time and live-cell imaging, as well as for patient blood or tissue samples.
Electrochemical Methods: This method measures the redox reaction of NAD/NADH on an electrode surface, generating a current proportional to the concentration of NAD/NADH. It can be used for rapid and point-of-care testing from patient samples.
NMR (Nuclear Magnetic Resonance) Spectroscopy: NMR utilizes magnetic fields and radio waves to detect and quantify NAD and its metabolites based on their specific spectral properties. NMR provides detailed information on the metabolic status of tissues, through sophisticated instrumentation. Examples include 1H-NMR or 31P-NMR spectroscopy for NAD quantification.

### 3. Therapeutic Agents and Targets

Therapeutic agents for use in the disclosed methods for treatment of the disclosed subjects are provided. The therapeutic agents are typically administered to a subject in an effective amount to treat the disease or disorder of the subject. The therapeutic agent can be in a pharmaceutical composition.

The therapeutic agent is most typically a compound that reduces the biological activity of a target molecule. Thus, compounds for decreasing the bioactivity of target molecules, and formulations formed therewith are provided. In some forms, the compound is an inhibitory polypeptide such as, but not limited to, an antibody; a small molecule or peptidomimedic, or an inhibitory nucleic acid that targets genomic or expressed nucleic acids (e.g., mRNA) encoding the target molecule, or a vector that encodes an inhibitory nucleic acid. The compound can reduce the expression or bioavailability of the target molecule. The inhibition can be competitive, non-competitive, uncompetitive, or product inhibition. Thus, an inhibitor can directly inhibit the target molecule, an inhibitor can inhibit another factor in a pathway that leads to induction, persistence, or amplification of the target molecule's expression, or a combination thereof. Thus, the therapeutic agents can be and are also referred to herein as inhibitors.

In some forms, the therapeutic agent is a protein binder that specifically binds to the target molecule, or a ligand or receptor thereof important for activity of the target molecule. In some forms, the protein binder is an antibody. Antibodies include not only intact antibodies, but also antibody fragments and antigen-binding components thereof, and fusion proteins including antigen binding fragments that are capable of immuno-specifically binding to the target molecule (or its counterpart ligand or receipt). The antibodies can be a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgD antibody, an IgE antibody, an IgM antibody, an IgG1 antibody, an IgG2 antibody, an IgG3 antibody, and an IgG4 antibody, or a fragment thereof, and fusion proteins formed therefrom. The antibodies and antigen binding fragments can be monospecific, bispecific, trispecific or multispecific.

The inhibitor can be a functional nucleic acid. Functional nucleic acids are nucleic acid molecules that have a specific function, such as binding a target molecule or catalyzing a specific reaction. As discussed in more detail below, functional nucleic acid molecules can be divided into the following non-limiting categories: antisense molecules, siRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Therefore, the compositions can include one or more functional nucleic acids designed to reduce expression of the target molecule's gene, or a gene product thereof. For example, the functional nucleic acid or polypeptide can be designed to target and reduce or inhibit expression or translation of target molecule's mRNA; or to reduce or inhibit expression, reduce activity, or increase degradation of target molecule protein. In some forms, the composition includes a vector suitable for in vivo expression of the functional nucleic acid.

Examples of functional nucleic acids include, but are not limited to, antisense oligonucleotides, siRNA, shRNA, miRNA, external guide sequences. External guide sequences (EGSs), ribozymes, aptamers, and CRISPR/Cas technology.

In some forms, the target molecule is P2X7 receptor. The P2X7 receptor is a type of purinergic receptor, which is a class of receptors activated by the molecule ATP (adenosine triphosphate). It is an ion channel found on the surface of various cell types, including immune cells such as macrophages and microglia. The P2X7 receptor plays a crucial role in several physiological and pathological processes, including inflammation, cell death, and the release of pro-inflammatory cytokines. Upon activation, the P2X7 receptor forms a non-selective ion channel that allows the flow of cations, such as calcium (Ca2+), sodium (Na+), and potassium (K+). The P2X7 receptor is heavily involved in the immune response. Its activation can lead to the release of pro-inflammatory cytokines like IL-1β and IL-18, and it is also implicated in the process of pyroptosis, a form of programmed cell death associated with inflammation. The P2X7 receptor is associated with various diseases, particularly those involving chronic inflammation, neurodegenerative conditions. For example, it has been implicated in rheumatoid arthritis, inflammatory bowel disease, or multiple sclerosis. Due to its role in inflammation and disease, the P2X7 receptor can be a target for therapeutic intervention.

The interaction between the P2X7 receptor and NAD (nicotinamide adenine dinucleotide) involves an interplay within cellular signaling, particularly in the immune system. Here are the key points about this interaction. Extracellular NAD can influence immune responses through the activation of P2X7 receptor. One of the aspects of NAD signaling is its involvement in the ADP-ribosylation of the P2X7 receptor. This process is catalyzed by ectoenzymes such as ART2 (ADP-ribosyltransferase 2) found on the surface of immune cells. ADP-ribosylation modifies the P2X7 receptor, enhancing its sensitivity to ATP. This modification can lead to the activation of the receptor even in the presence of lower concentrations of extracellular ATP, which would not normally be sufficient to activate the receptor. This process is particularly relevant in T cells and other immune cells. The NAD-induced activation of the P2X7 receptor plays a role in the regulation of immune cell functions. This includes the promotion of pro-inflammatory responses, cell death, and the release of cytokines. This mechanism can contribute to the regulation of immune responses during inflammation and infection.

In some forms, the inhibitor of P2X7 receptor is A438079. A438079 as a selective small-molecule antagonist of P2X7 receptor is known in the art.

In some forms, the target molecule is NAMPT or genes expressing NAMPT. NAMPT is a protein that catalyzes the condensation of nicotinamide with 5-phosphoribosyl-1-pyrophosphate to yield nicotinamide mononucleotide, one step in the biosynthesis of nicotinamide adenine dinucleotide. The protein belongs to the nicotinic acid phosphoribosyltransferase (NAPRTase) family and is involved in many biological processes, including metabolism, stress response and aging. Nicotinamide phosphoribosyltransferase (NAMPT) is a regulator of the intracellular nicotinamide adenine dinucleotide (NAD) pool and, thus, regulates the activity of NAD-dependent enzymes.

### 4. Combination Therapy

The disclosed methods for treating HCC can be used in combination with an immune checkpoint inhibitor.

In some forms, the methods administer modified T cells and/or a monoclonal antibody in combination with other therapeutic agents or treatment modalities. Any of the disclosed pharmaceutical compositions including modified cells, such as therapeutic T cells can be used alone, or in combination with other therapeutic agents or treatment modalities, for example, chemotherapy or stem-cell transplantation. As used herein, “combination” or “combined” refer to either concomitant, simultaneous, or sequential administration of the therapeutics.

In some forms, the pharmaceutical compositions and other therapeutic agents are administered separately through the same route of administration. In other forms, the pharmaceutical compositions and other therapeutic agents are administered separately through different routes of administration. The combinations can be administered either concomitantly (e.g., as an admixture), separately but simultaneously (e.g., via separate intravenous lines into the same subject; one agent is given orally while the other agent is given by infusion or injection, etc.,), or sequentially (e.g., one agent is given first followed by the second).

The compositions and methods described herein may be used as a first therapy, second therapy, third therapy, or combination therapy with other types of therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

The disclosed pharmaceutical compositions and/or other therapeutic agents, procedures or modalities can be administered during periods of active disease, or during a period of remission or less active disease. The pharmaceutical compositions can be administered before the additional treatment, concurrently with the treatment, post-treatment, or during remission of the disease or disorder. When administered in combination, the disclosed pharmaceutical compositions and the additional therapeutic agents (e.g., second or third agent), or all, can be administered in an amount or dose that is higher, lower or the same than the amount or dosage of each agent used individually, e.g., as a monotherapy. In certain forms, the administered amount or dosage of the disclosed pharmaceutical composition, the additional therapeutic agent (e.g., second or third agent), or all, is lower (e.g., at least 20%, at least 30%, at least 40%, or at least 50%) than the amount or dosage of each agent used individually, e.g., as a monotherapy (e.g., required to achieve the same therapeutic effect).

Examples of preferred additional therapeutic agents include other conventional therapies known in the art for treating the desired disease, disorder or condition. In some forms, the therapeutic agent is one or more other targeted therapies (e.g., a targeted cancer therapy) and/or immune-checkpoint blockage agents (e.g., anti LAG-3, anti CTLA 4, anti PD1, and/or anti PDL1 agents such as antibodies).

Immunotherapy is a type of medical treatment that harnesses the body's own immune system to combat diseases, particularly cancer and certain autoimmune disorders. The goal of immunotherapy is to stimulate or enhance the body's natural defenses against harmful cells or substances, such as cancer cells or pathogens. There are different approaches to immunotherapy, but one of the common forms of immunotherapy include immune checkpoint inhibitors.

Immune checkpoints refer to inhibitory pathways hardwired into the immune system that are crucial for maintaining self-tolerance and modulating the duration and amplitude of physiological immune responses in peripheral tissues in order to minimize collateral tissue damage. Immune checkpoint molecules can be stimulatory or inhibitory to an immune checkpoint. The present disclosure and claims refer to inhibitory molecules of immune checkpoints as “immune checkpoint molecules.”

Agents that block immune checkpoint molecules (e.g. PD-1, PD-L1, CTLA-4, or LAG-3), suggest opportunities to enhance antitumor immunity with the ability to produce effective clinical responses.

An immune checkpoint inhibitor is a type of drug that blocks the signaling of immune checkpoint molecule(s) made by some types of immune system cells, such as T cells and some cancer cells. Immune checkpoint inhibitors therefore can cause immune checkpoint blockade. Immune checkpoint molecules (e.g., PD-1) help keep immune responses in check and can keep T cells from killing cancer cells. When these molecules are blocked, the “brakes” on the immune system are released (inhibition of the immune system is reduced or blocked) and T cells are able to kill cancer cells better. Examples of checkpoint proteins found on T cells or cancer cells include PD-1, PD-L1, CTLA-4, and LAG-3. In some embodiments, immune checkpoint molecules are proteins. In some embodiments, immune checkpoint molecules are nucleic acids that encode the proteins. In some embodiments, immune checkpoint inhibitors bind to and/or antagonize immune checkpoint molecules.

The agent that binds to and/or antagonizes an immune checkpoint molecule is an immune checkpoint inhibitor. One or more immune checkpoint inhibitors refer to one or more different inhibitors. Each different inhibitor has a different molecular structure. Two different inhibitors may bind the same immune checkpoint molecule, or each may bind a different immune checkpoint molecule.

An inhibitor or antagonist, as used herein, is a molecule that inhibits, reduces, or blocks activity of an immune checkpoint molecule to inhibit a suppressive effect that the immune checkpoint molecule has on the immune system. The inhibitor or antagonist can directly bind the immune checkpoint molecule, a molecule controlling the expression of the immune checkpoint molecule, or a ligand of the immune checkpoint molecule that mediates the activity of the immune checkpoint molecule. The inhibitor or antagonist may be an antibody (including a humanized antibody), a small molecule, a peptide, or a nucleic acid (e.g., an antisense molecule, or a single- or double-stranded RNAi molecule). Activity of the immune checkpoint molecule is referred to as its suppressive effect on an immune checkpoint. An immune checkpoint inhibitor can reduce or block the activity of an immune checkpoint molecule.

In some forms, the immune checkpoint inhibitor comprises a therapeutic antibody. In some forms, the immune checkpoint inhibitor targets programmed cell death ligand 1 (PD-L1). In some forms, the immune checkpoint inhibitor comprises a therapeutic antibody, and the therapeutic antibody is avelumab, atezolizumab, durvalumab, envafolimab, cosibelimab, or AUNP-12.

In some forms, the immune checkpoint inhibitor targets programmed cell death protein 1 (PD-1). In some forms, the immune checkpoint inhibitor comprises a therapeutic antibody, and the therapeutic antibody is pembrolizumab, nivolumab, cemiplimab, spartalizumab, camrelizumab, sintilimab, tislelizumab, toripalimab, dostarlimab, retifanlimab, AMP-224, or MEDI0680.

In some forms, the immune checkpoint inhibitor targets cytotoxic T-lymphocyte-associated protein 4 (CTLA-4). In some forms, the immune checkpoint inhibitor comprises a therapeutic antibody, and the therapeutic antibody is ipilimunab or tremelimumab.

In some forms, the immune checkpoint inhibitor targets lymphocyte-activation gene 3 (LAG-3). In some forms, the immune checkpoint inhibitor comprises a therapeutic antibody, and the therapeutic antibody is relatimab.

i. Immunotherapy Targets
a. Programmed Cell Death Ligand 1 (PD-L1)

In humans, programmed death-ligand 1 (PD-L1), also known as B7 homolog 1 (B7-H1) or cluster of differentiation 274 (CD274), is a 40 kDa type 1 transmembrane protein that is encoded by the CD274 gene. Foreign antigens normally induce an immune response triggering proliferation of antigen-specific T cells, such as antigen-specific CD8+ T cells. PD-L1 is an immune checkpoint inhibitor that may block or lower such an immune response. PD-L1 may play a major role in suppressing the immune system during events such as pregnancy, tissue allografts, autoimmune disease, and other disease states, such as hepatitis and cancer. The PD-L1 ligand binds to its receptor, PD-1, found on activated T cells, B cells, and myeloid cells, thereby modulating activation or inhibition. In addition to PD-1, PD-L1 also has an affinity for the costimulatory molecule CD80 (B7-1).

PD-L1 Antagonist. A PD-L1 antagonist, as used herein, is a molecule that binds to PD-L1 protein or to a gene or nucleic acid encoding PD-L1 protein and inhibits or prevents PD-1 activation. Without wishing to be bound by theory, it is believed that such molecules reduce or block the interaction of PD-L1 with PD-1. PD-L1 activity may be blocked by molecules that selectively bind to and block the activity of PD-L1. Anti-PD-L1 antibodies block interactions between PD-L1 and both PD-1 and B7-1 (also known as CD80).

Block means inhibit or prevent the transmission of an inhibitory signal mediated via such PD-L1 binding. PD-L1 antagonists include, for example: BMS-936559, also known as MDX-1105 (Bristol-Meyers Squibb), a fully human, high affinity, immunoglobulin (Ig) G4 monoclonal antibody to PD-L; MPDL3280A, also known as RG7446 or atezolizumab (Genentech/Roche), an engineered human monoclonal antibody targeting PD-L1;MSB0010718C, also known as avelumab (Merck), a fully human IgGI monoclonal antibody that binds to PD-L1; and MEDI473 (AstraZeneca/MedImmune), a human immunoglobulin (Ig) Glk monoclonal antibody that blocks PD-L1 binding to its receptors.

Agents that bind to the DNA or mRNA encoding PD-L1 also can act as PD-L inhibitors, e.g., small inhibitory anti-PD-L1 RNAi, small inhibitory anti-PD-L1 RNA, anti-PD-L1 anti-sense RNA, or dominant negative PD-L1 protein. Antagonists of or agents that antagonize PD-L1, e.g., anti-PD-L1 antibodies and PD-L1 antagonists, may include, but are not limited to those previously mentioned and any of those that are disclosed in Stewart et al., 2015, 3 (9): 1052-62; Herbst et al., 2014, Nature Volume: 515: Pages: 563-567; Brahmer et al., N Engl J Med 2012; 366:2455-2465; U.S. Pat. No. 8,168,179; US20150320859; and/or US20130309250 all incorporated herein by reference. In clinical trials, treatment with anti-PD-L1 antibodies resulted in less adverse events than did treatment with anti-PD-1 antibodies (Shih et al.,2014). In some embodiments, anti-PD-L1 inhibitors be used for treatment in combination with additional immune checkpoint blockade, e.g., with anti-PD-1, anti-CTLA-4, and/oranti-LAG-3 inhibitors.

b. Programmed Cell Death Protein 1 (PD-1)

In humans, programmed cell death protein 1 (PD-1) is encoded by the PDCD1 gene. PDCD1 has also been designated as CD279 (cluster of differentiation 279). This gene encodes a cell surface membrane protein of the immunoglobulin superfamily. PD-1 is a 288 amino acid cell surface protein molecule. PD-1 is expressed on the surface of activated T cells, B cells, and macrophages. PD-1 is expressed in pro-B cells and is thought to play a role in their differentiation. See T. Shino hara et al., Genomics 23 (3): 704-6 (1995). PD-1 is a member of the extended CD28CTLA-4 family of T cell regulators. (Y. Ishida et al., “EMBO J. 11 (11): 3887-95, (1992)). PD-1 may negatively regulate immune responses. PD-1 limits autoimmunity and the activity of T cells in peripheral tissues at the time of an inflammatory response to infection.

PD-1 has two ligands, PD-L1 and PD-L2, which are members of the B7 family. PD-L1 protein is upregulated on macrophages and dendritic cells (DC) in response to lipopolysaccharide (LPS) and granulocyte-macrophage colony-stimulating factor (GM-CSF) treatment, and on T cells and B cells upon T cell receptor (TCR) and B cell receptor signaling, whereas in resting mice, PD-L1 mRNA can be detected in the heart, lung, thymus, spleen, and kidney. PD-L1 is expressed on almost all murine tumor cell lines, including PA myeloma, PH815 mastocytoma, and B16 melanoma upon treatment with IFN-y. PD-L1 has been found to be highly expressed by several cancers and several PD-1 antagonists are being developed or are approved for treatment of cancer. PD-L2 expression is more restricted and is expressed mainly by DCs and a few tumor lines.

Programmed Death 1 (PD-1) antagonist. A PD-1 antagonist is a molecule that binds to PD-1 protein or to a gene or nucleic acid encoding PD-1 protein and inhibits or prevents PD-1 activation. Without wishing to be bound by theory, it is believed that such molecules reduce or block the interaction of PD-1 with its ligand(s) PD-L1 and/or PD-L2. PD-1 activity may be interfered with by antibodies that bind selectively to and block the activity of PD-1. The activity of PD-1 can also be inhibited or blocked by molecules other than antibodies that bind PD-1. Such molecules can be small molecules or can be peptide mimetics of PD-L1 and PD-L2 that bind PD-1 but do not activate PD-1. Molecules that antagonize PD-1 activity include those described in U.S. Publications 20130280265, 20130237580, 20130230514, 20130109843, 20130108651, 20130017199, 20210008200 and 20120251537, 20110271358, EP 2170959B1, the entire disclosures of which are incorporated herein by reference. See also M. A. Curran, et al., Proc. Natl. Acad. Sci. USA 107, 4275 (2010); S. L. Topalian, et al., New Engl. J. Med.366, 2443 (2012); J. R. Brahmer, et al., New Engl. J. Med. 366, 2455 (2012); and D. E. Dolan et al., Cancer Control 21, 3 (2014), all incorporated by reference herein, in their entireties.

Herein, exemplary PD-1 antagonists include: nivolumab, also known as BMS-936558, OPDIVO® (Bristol-Meyers Squibb, and also known as MDX-1106 or ONO 4538), a fully human IgG4 monoclonal antibody against PD-1; pidilizumab, also known as CT-011 (CureTech), a humanized IgG1 monoclonal antibody that binds PD-1; MK-3475 (Merck, and also known as SCH 900475), an IgG4 antibody that binds PD-1; and pembrolizumab (Merck, also known MK-3475, lambrolizumab, KEYTRUDA®), a humanized IgG4-kappa monoclonal antibody that binds PD-1; MEDI-0680 (AstraZeneca/MedImmune), a monoclonal antibody that binds PD-1; and REGN2810 (Regeneron/Sanofi), a monoclonal antibody that binds PD-1. Another exemplary PD-1 antagonist is AMP 224 (Glaxo Smith Kline and Amplimmune), a recombinant fusion protein composed of the extracellular domain of the PD-1 ligand programmed cell death ligand 2 (PD-L2) and the Fc region of human IgG1, that binds to PD-1. Agents that interfere and bind to the DNA or mRNA encoding PD-1 also can act as PD-1 inhibitors. Examples include a small inhibitory anti-PD-1 RNAi, an anti-PD-1 antisense RNA, or a dominant negative protein. PDL-2 fusion protein AMP-224 (codeveloped by Glaxo Smith Kline and Amplimmune) is believed to bind to and block PD-1. In some embodiments, anti-PD-1 inhibitors may be used for treatment in combination with additional immune checkpoint blockade, e.g., with anti-PD-L1, anti CTLA-4, and/or anti-LAG-3 inhibitors.

c. Cytotoxic T-Lymphocyte-Associated Protein 4 (CTLA-4)

CTLA-4 (also known as CTLA-4 or cluster of differentiation 152 (CD152)), is a transmembrane glycoprotein that, in humans, is encoded by the CTLA-4 gene. CTLA-4 is a member of the immunoglobulin superfamily, which is expressed on the surface of helper T cells and is present in regulatory T cells, where it may be important for immune function. CTLA-4, like the homologous CD28, binds to B7 molecules, particularly CD80/B7-1 and CD86/B7-2 on antigen-presenting cells (APCs), thereby sending an inhibitory signal to T cells. CTLA-4 functions as an immune checkpoint that inhibits the immune system and is important for maintenance of immune tolerance.

CTLA-4 antagonist. A CTLA-4 antagonist, as used herein, is a molecule that binds to CTLA-4 protein or to a gene or nucleic acid encoding CTLA-4 protein and inhibits or prevents CTLA-4 activation. Without wishing to be bound by theory, it is believed that such molecules reduce or block the interaction of CTLA-4 with its ligands, e.g., B7 molecules CD80/B7-1 and CD86/B7-2. CTLA-4 activity may be blocked by molecules that bind selectively to and block the activity of CTLA-4 or that bind selectively to its counter-receptors, e.g., CD80, CD86, etc. and block activity of CTLA-4. Blocking means inhibit or prevent the transmission of an inhibitory signal via CTLA-4. In some embodiments, anti-CTLA-4 antibodies may be used for treatment in combination with additional immune checkpoint blockade, e.g., with anti-PD-1, anti-PD-L1, and/oranti-LAG-3 treatment. CTLA-4 antagonists include, for example, inhibitory antibodies directed to CD80, CD86, and/or CTLA-4; small molecule inhibitors of CD80, CD86, and CTLA-4; antisense molecules directed against CD80, CD86, and/or CTLA-4; adnectins directed against CD80, CD86, and/or CTLA-4; and RNAi inhibitors (both single and double stranded) of CD80, CD86, and/or CTLA-4. Suitable CTLA-4 antagonists and/or anti-CTLA-4 antibodies include humanized anti-CTLA-4 antibodies, such as MDX-010/ipilimumab (Bristol-Meyers Squibb), tremelimumab/CP-675,206 (Pfizer, AstraZeneca), and antibodies that are disclosed in PCT Publication No. WO 2001/014424, PCT Publication No. WO 2004/035607, U.S. Publication No. 2005/0201994, European Patent No. EP 1212422 B1, U.S. Pat. Nos. 5,811,097, 5,855,887, 6,051,227, 6,984,720, 7,034,121, 8,475,790, U.S. Publication Nos. 2002/0039581 and/or2002/086014, the entire disclosures of which are incorporated herein by reference.

d. Lymphocyte-Activation Gene 3 (LAG-3)

The term “LAG-3” or “LAG3” refers to Lymphocyte Activation Gene-3. The LAG-3 protein, which belongs to immunoglobulin (lg) superfamily, comprises a 503-amino acid type I transmembrane protein with four extracellular lg-like domains, designated DI to D4. LAG-3 is an immune checkpoint protein that plays a role in regulating the immune system. As described herein, the term LAG-3 includes variants, isoforms, homologs, orthologs, and paralogs. For example, antibodies specific for a human LAG-3 protein may, in certain cases, cross-react with a LAG-3 protein from a species other than human. In other embodiments, the antibodies specific for a human LAG-3 protein may be completely specific for the human LAG-3 protein and may not exhibit species or other types of cross-reactivity or may cross-react with LAG-3 from certain other species but not all other species (e.g., cross-react with monkey LAG-3, but not mouse LAG-3). The term “human LAG-3” refers to human sequence LAG-3, such as the complete amino acid sequence of human LAG-3 having GenBank Accession No. NP 002277. LAG-3 is also known in the art as, for example, CD223. The human LAG-3 sequence may differ from human LAG-3 of GenBank Accession No. NP 002277 by having, e.g., conserved mutations or mutations in non-conserved regions and the

LAG-3 has substantially the same biological function as the human LAG-3 of GenBank Accession No. NP 002277. For example, a biological function of human LAG-3 is having an epitope in the extracellular domain of LAG-3 that is specifically bound by an antibody of the instant disclosure or a biological function of human LAG-3 is binding to MHC Class II molecules.

LAG-3 Antagonists. Inhibitors of LAG-3 are immunotherapy treatments for various cancers and autoimmune diseases. One of the notable LAG-3 inhibitors that has been developed is Relatlimab. Relatlimab is a monoclonal antibody that targets LAG-3 and is designed to block its interaction with its ligand, MHC class II molecules, which can suppress immune responses. By inhibiting LAG-3, Relatlimab aims to enhance the activation and function of T cells, the immune cells responsible for recognizing and attacking cancer cells. Agents that interfere and bind to the DNA or mRNA encoding LAG-3 proteins also can act as LAG-3 inhibitors. Examples include a small inhibitory anti-LAG-3 RNAi, an anti-LAG-3antisense RNA. In some embodiments, anti-LAG3 inhibitors may be used for treatment in combination with additional immune checkpoint blockade, e.g., with anti-PD-1, anti-PD-L1, and/or anti-CTLA-4 inhibitors.

### 5. Formulations

The disclosed compounds can be formulated in a pharmaceutical composition. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration.

The compositions can be administered systemically.

Drugs can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are typically prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, disintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6^{th} Edition, Ansel et.al., (Media, PA: Williams and Wilkins, 1995) which provides information on carriers, materials, equipment and process for preparing tablets and capsules and delayed release dosage forms of tablets, capsules, and granules.

The compound can be administered to a subject with or without the aid of a delivery vehicle. Appropriate delivery vehicles for the compounds are known in the art and can be selected to suit the particular active agent. For example, in some forms, the active agent(s) is incorporated into or encapsulated by, or bound to, a nanoparticle, microparticle, micelle, synthetic lipoprotein particle, or carbon nanotube. For example, the compositions can be incorporated into a vehicle such as polymeric particles which provide controlled release of the active agent(s). In some forms, release of the drug(s) is controlled by diffusion of the active agent(s) out of the particles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing particles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some forms, both agents are incorporated into the same particles and are formulated for release at different times and/or over different time periods. For example, in some forms, one of the agents is released entirely from the particles before release of the second agent begins. In other forms, release of the first agent begins followed by release of the second agent before the all of the first agent is released. In still other forms, both agents are released at the same time over the same period of time or over different periods of time.

i. Formulations for Parenteral Administration

Compounds and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

ii. Oral Immediate Release Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to,, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

iii. Extended Release Dosage Forms

The extended release formulations are generally prepared as diffusion or osmotic systems, for example, as described in “Remington—The science and practice of pharmacy” (20th ed., Lippincott Williams & Wilkins, Baltimore, MD, 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form having single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

iv. Delayed Release Dosage Forms

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma; Westerstadt, Germany), including EUDRAGIT®. L30D-55 and L100-55 (soluble at pH 5.5 and above), EUDRAGIT®. L-100 (soluble at pH 6.0 and above), EUDRAGIT®. S (soluble at pH 7.0 and above, as a result of a higher degree of esterification), and EUDRAGITS®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, PA: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

v. Topical and Transdermal Formulations

Transdermal formulations may also be prepared. These will typically be gels, ointments, lotions, sprays, or patches, all of which can be prepared using standard technology. Transdermal formulations can include penetration enhancers.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular forms, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one form, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one form, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one form, the emulsifier is glycerol stearate.

A “lotion” is a low-to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one form, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one form, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) composed of the drug dispersed or dissolved in a mixture of surfactant(s) and lipophillic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components.

Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred form, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Additional agents that can be added to the formulation include penetration enhancers. In some forms, the penetration enhancer increases the solubility of the drug, improves transdermal delivery of the drug across the skin, in particular across the stratum corneum, or a combination thereof. Some penetration enhancers cause dermal irritation, dermal toxicity and dermal allergies. However, the more commonly used ones include urea, (carbonyldiamide), imidurea, N, N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly(21)oxyethylene ether) (ICI Americas Inc. Corp.). Chemical penetrations and methods of increasing transdermal drug delivery are described in Inayat, et al., Tropical Journal of Pharmaceutical Research, 8(2):173-179 (2009) and Fox, et al., Molecules, 16:10507-10540 (2011). In some forms, the penetration enhancer is, or includes, an alcohol such ethanol, or others disclosed herein or known in the art.

Delivery of drugs by the transdermal route has been known for many years. Advantages of a transdermal drug delivery compared to other types of medication delivery such as oral, intravenous, intramuscular, etc., include avoidance of hepatic first pass metabolism, ability to discontinue administration by removal of the system, the ability to control drug delivery for a longer time than the usual gastrointestinal transit of oral dosage form, and the ability to modify the properties of the biological barrier to absorption.

Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir; matrix or monolithic devices are typically characterized by a falling drug flux with time, as the matrix layers closer to the skin are depleted of drug. Usually, reservoir patches include a porous membrane covering the reservoir of medication which can control release, while heat melting thin layers of medication embedded in the polymer matrix (e.g., the adhesive layer), can control release of drug from matrix or monolithic devices. Accordingly, the active agent can be released from a patch in a controlled fashion without necessarily being in a controlled release formulation.

Patches can include a liner which protects the patch during storage and is removed prior to use; drug or drug solution in direct contact with release liner; adhesive which serves to adhere the components of the patch together along with adhering the patch to the skin; one or more membranes, which can separate other layers, control the release of the drug from the reservoir and multi-layer patches, etc., and backing which protects the patch from the outer environment.

Common types of transdermal patches include, but are not limited to, single-layer drug-in-adhesive patches, wherein the adhesive layer contains the drug and serves to adhere the various layers of the patch together, along with the entire system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for control release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described in U.S. Pat. Nos. 6,461,644, 6,676,961, 5,985,311, and 5,948,433.

### 6. Dosage Units and Methods of Administration

A treatment regimen can include one or multiple administrations of the compositions including an effective amount of one or more of the compounds for achieving a desired physiological change, including administering to an animal, such as a mammal, especially a human being, an effective amount of the compositions to treat the disease or symptom thereof, or to produce the physiological change.

The effective amount or therapeutically effective amount of a pharmaceutical compositions can be a dosage sufficient to treat, inhibit, or alleviate one or more symptoms of a disease or disorder or to otherwise provide a desired pharmacologic and/or physiologic effect, for example, reducing, inhibiting, or reversing one or more of the underlying pathophysiological mechanisms underlying a disease or disorder, such as PCAD. In preferred forms, the desired physiological change could include improvement in one or more symptoms of a disease or condition treated herein, such as improvement in breathing and exercise capacity or improved sensitivity to irritants or drop in albuterol use or improved vocal cord function or reduction in cough in the subject.

In some forms, when administrating the pharmaceutical composition, the amount administered can be expressed as the amount effective to achieve a desired effect in the recipient.

The effective amount of the pharmaceutical composition will vary based on the active agent and from subject to subject, depending on the species, age, weight and general condition of the subject, the severity of the disorder being treated, and its mode of administration. Thus, it is not possible to specify an exact amount for every pharmaceutical composition. However, an appropriate amount can be determined by one of ordinary skill in the art using only routine experimentation given the teachings herein. For example, effective dosages and schedules for administering the pharmaceutical composition can be determined empirically. In some forms, the dosage ranges for the administration of the composition are those large enough to resolve mucosal hyperreactivity throughout the respiratory tract.

Preferably, the dosage is not so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the disease to be treated. The dosage can be adjusted by the individual physician in the event of any counter-indications. It will also be appreciated that the effective dosage of the composition can increase or decrease over the course of a particular treatment. Changes in dosage can result and become apparent from the results of diagnostic assays.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can determine optimum dosages, dosing methodologies and repetition rates. Optimum dosages can vary depending on the relative potency of individual pharmaceutical compositions, and can generally be estimated based on EC50s found to be effective in in vitro and in vivo animal models.

In cases of a solid dosage form, examples of daily dosages of the compounds described herein which can be used are an effective amount within the dosage range of about 0.001 mg to about 2 mg per kilogram of body weight, about 0.001 mg to about 5 mg per kilogram of body weight, about 0.001 mg to about 10 mg per kilogram of body weight, about 0.001 mg to about 20 mg per kilogram of body weight, about 0.001 mg to about 50 mg per kilogram of body weight, about 0.001 mg to about 100 mg per kilogram of body weight, about 0.001 mg to about 200 mg per kilogram of body weight, or about 0.001 mg to about 300 mg per kilogram of body weight.

When administered orally or by inhalation, examples of daily dosages are an effective amount within the dosage range of about 0.1 mg to about 10 mg, or about 0.1 mg to about 20 mg, or about 0.1 mg to about 30 mg, or about 0.1 mg to about 40 mg, or about 0.1 mg to about 50 mg, or about 0.1 mg to about 60 mg, or about 0.1 mg to about 70 mg, or about 0.1 mg to about 80 mg, or about 0.1 mg to about 90 mg, or about 0.1 mg to about 100 mg, or about 0.1 mg to about 200 mg, or about 0.1 mg to about 300 mg, or about 0.1 mg to about 400 mg, or about 0.1 mg to about 500 mg, or about 0.1 mg to about 600 mg, or about 0.1 mg to about 700 mg, or about 0.1 mg to about 800 mg, or about 0.1 mg to about 900 mg, or about 0.1 mg to about 1 g, or about 20 mg to 300 mg, or about 20 mg to 500 mg, or about 20 mg to 700 mg, or about 20 mg to 1000 mg, or about 50 mg to 1500 mg, or about 50 mg to 2000 mg.

Exemplary fixed daily doses include about 1 mg, about 2 mg, about 3 mg, about 4 mg, about 5 mg, about 6 mg, about 7 mg, about 8 mg, about 9 mg, about 10 mg, about 12 mg, about 15 mg, about 18 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1200 mg, about 1500 mg, or about 2000 mg, independently of body weight. However, it is understood that pediatric patients may require smaller dosages, and depending on the severity of the disease and condition of the patient, dosages may vary.

When formulated as a liquid, the concentration of the compounds described herein may be about 0.01 mg/ml to about 0.1 mg/ml or about 0.1 mg/ml to about 1 mg/ml, but can also be about 1 mg/ml to about 10 mg/ml or about 10 mg/ml to about 100 mg/ml. The liquid formulation could be a solution or a suspension. When formulated as a solid, for example as a tablet or as a powder for inhalation, the concentration, expressed as the weight of a compound divided by total weight, will typically be about 0.01% to about 0.1%, about 0.1% to about 1%, about 1% to about 10%, about 10% to about 20%, about 20% to about 40%, about 40% to about 60%, about 60% to about 80%, or about 80% to about 100%.

In some forms, administration of the composition will be given as a long-term treatment regimen whereby pharmacokinetic steady state conditions will be reached.

For example, some forms, antibodies are packaged in a hermetically sealed container, such as an ampoule or sachette, indicating the quantity of antibody. In some forms, the antibodies are supplied as a dry sterilized lyophilized powder or water free concentrate in a hermetically sealed container and can be reconstituted, e.g., with water or saline to the appropriate concentration for administration to a subject. For example, antibodies can be supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, or at least 75 mg. The lyophilized antibodies can be stored at between 2 and 8° C. in their original container and the antibodies can be administered within 12 hours, preferably within 6 hours, within 5 hours, within 3 hours, or within 1 hour after being reconstituted. In an alternative form, antibodies can be supplied in liquid form in a hermetically sealed container indicating the quantity and concentration of the antibody, fusion protein, or conjugated molecule. Preferably, the liquid form of the antibodies are supplied in a hermetically sealed container at least 1 mg/ml, more preferably at least 2.5 mg/ml, at least 5 mg/ml, at least 8 mg/ml, at least 10 mg/ml, at least 15 mg/ml, at least 25 mg/ml, at least 50 mg/ml, at least 100 mg/ml, at least 150 mg/ml, at least 200 mg/ml of the antibodies.

For antibodies, the dosage administered to a patient is typically 0.01 mg/kg to 100 mg/kg of the patient's body weight. Preferably, the dosage administered to a patient is between 0.01 mg/kg and 20 mg/kg, 0.01 mg/kg and 10 mg/kg, 0.01 mg/kg and 5 mg/kg, 0.01 and 2 mg/kg, 0.01 and 1 mg/kg, 0.01 mg/kg and 0.75 mg/kg, 0.01 mg/kg and 0.5 mg/kg, 0.01 mg/kg to 0.25 mg/kg, 0.01 to 0.15 mg/kg, 0.01 to 0.10 mg/kg, 0.01 to 0.05 mg/kg, or 0.01 to 0.025 mg/kg of the patient's body weight. In particular, the invention contemplates that the dosage administered to a patient is 0.2 mg/kg, 0.3 mg/kg, 1 mg/kg, 3 mg/kg, 6 mg/kg or 10 mg/kg. A dose as low as 0.01 mg/kg may show appreciable pharmacodynamic effects. Dose levels of 0.10-1 mg/kg are predicted to be most appropriate. Higher doses (e.g., 1-30 mg/kg) are also contemplated. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies may be reduced by enhancing uptake and tissue penetration of the antibodies by modifications such as, for example, lipidation.

Injections and infusion of the disclosed compositions can be repeated as often and as many times as the patient can tolerate until the desired response is achieved. Thus, antibodies can also be administered once or multiple times at these dosages. The optimal dosage and treatment regime for a particular patient can be determined by one skilled in the art of medicine by monitoring the patient for signs of disease and adjusting the treatment accordingly. In some forms, the unit dosage is in a unit dosage form for intravenous injection. In some forms, the unit dosage is in a unit dosage form for oral administration. In some forms, the unit dosage is in a unit dosage form for inhalation. In some forms, the unit dosage is in a unit dosage form for subcutaneous injection.

Treatment can be continued for an amount of time sufficient to achieve one or more desired therapeutic goals. The timing of the administration of the composition will also depend on the formulation and/or route of administration used. The compound may be administered once daily, but may also be administered two, three or four times daily, or every other day, or once or twice per week. For example, the subject can be administered one or more treatments 1, 2, 3, 4, 5, 6, 7, 8, 9, 10, 11, 12, 13, 14, 15, 16, 17, 18, 19, 20, 21, 22, 23, or 24 hours, days, weeks, or months apart.

In some forms, the compositions are formulated for extended release. For example, the formulation can be suitable for administration once daily or less. In some forms, the composition is only administered to the subject once every 24-48 hours.

Treatment can be continued for a desired period of time, and the progression of treatment can be. In some forms, administration is carried out every day of treatment, or every week, or every fraction of a week. In some forms, treatment regimens are carried out over the course of up to two, three, four or five days, weeks, or months, or for up to 6 months, or for more than 6 months, for example, up to one year, two years, three years, or up to five years.

The efficacy of administration of a particular dose of the pharmaceutical compositions can be determined by evaluating the aspects of the medical history, signs, symptoms, and objective laboratory tests that are known to be useful in evaluating the status of a subject in need for the treatment of a disease or condition discussed herein. These signs, symptoms, and objective laboratory tests will vary, depending upon the particular disease or condition being treated or prevented, as will be known to any clinician who treats such patients or a researcher conducting experimentation in this field.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

The disclosed invention can be further understood by the following numbered paragraphs:
Paragraph 1. A method of treating a subject in need thereof, the method comprising administering to the subject an effective amount of:
   (i) an inhibitor of P2X7 receptor,
   (ii) an inhibitor of nicotinamide phosphoribosyltransferase (NAMPT), or
   (iii) a combination thereof.
Paragraph 2. The method of paragraph 1, wherein the subject has hepatocellular carcinoma (HCC) or has been identified as being at increased risk of developing HCC.
Paragraph 3. The method of paragraph 2, wherein, prior to the administering, the subject has HCC and is determined to be substantially unresponsive to immune checkpoint inhibitors.
Paragraph 4. The method of paragraph 2, wherein, prior to the administering, the subject (a) has HCC and (b) has elevated nicotinamide adenine dinucleotide (NAD) levels when treated with an immune checkpoint inhibitor relative to average NAD levels of HCC patients when treated with an immune checkpoint inhibitor.
Paragraph 5. The method of paragraph 4, wherein the elevated NAD levels are detected in serum and/or tissues of the subject.
Paragraph 6. The method of any one of paragraphs 1-5 further comprising treating the subject with an immune checkpoint inhibitor at the same time or after the administering.
Paragraph 7. The method of paragraph 6, wherein the administering is in combination with the immune checkpoint inhibitor.
Paragraph 8. The method of paragraph 6 or paragraph 7, wherein the immune checkpoint inhibitor targets programmed cell death protein 1 (PD-1), programmed cell death ligand 1 (PD-L1), cytotoxic T-lymphocyte-associated protein 4 (CTLA-4), lymphocyte-activation gene 3 (LAG-3), or combination thereof.
Paragraph 9. The method of any one of paragraphs 1-8, wherein the inhibitor of P2X7 receptor is a polypeptide, a small molecule, a peptidomimetic, a nucleic acid that targets genomic or expressed nucleic acids encoding the P2X7 receptor, or a vector that encodes an inhibitory nucleic acid that targets genomic or expressed nucleic acids encoding the P2X7 receptor.
Paragraph 10. The method of paragraph 9, wherein the polypeptide is an antibody.
Paragraph 11. The method of paragraph 9, wherein the expressed nucleic acid is mRNA.
Paragraph 12. The method of any one of paragraphs 1-9, wherein the inhibitor of P2X7 receptor is A438079, A-740003, AZ-10606120, or a derivative thereof.
Paragraph 13. The method of any one of paragraphs 1-12, wherein the inhibitor of NAMPT is a polypeptide, a small molecule, a peptidomimetic, a nucleic acid that targets genomic or expressed nucleic acids encoding NAMPT, or a vector that encodes an inhibitory nucleic acid that targets genomic or expressed nucleic acids encoding NAMPT.
Paragraph 14. The method of paragraph 13, wherein the polypeptide is an antibody.
Paragraph 15. The method of paragraph 13, wherein the expressed nucleic acid is mRNA.
Paragraph 16. The method of any one of paragraphs 1-13, wherein the NAMPT inhibitor is a functional nucleic acid selected from the group consisting of an antisense molecule, siRNA, miRNA, aptamer, ribozyme, triplex forming molecule, RNAi, and external guide sequence.
Paragraph 17. The method of paragraph 13, wherein the small molecule is FK866, GMX1778, or a derivative thereof.

### EXAMPLES

### Methods

Human HCC data. Bulk RNA sequencing data of 373 human HCC samples was obtained from The Cancer Genome Atlas (TCGA) via cBioportal (http://www.cbioportal.org/). CD8+ T cells and NK cells proportion in tumors of these samples was estimated using CIBERSORTx (https://cibersortx.stanford.edu/) and their correlations with NAMPT/P2RX7 expressions were investigated. Spatial transcriptomic data of HCC samples (anti-PD-1 responder and non-responder) was obtained from SpatialTME (https://www.spatialtme.yelab.site/).

Cell culture and cell lines establishment. Three human HCC cell lines and a mouse HCC cell line were used in this study. Huh7 was kind gift from Dr Z. Y. Yang (Fudan University). Hep3B, HepG2 and Hepa1-6 cells were purchased from American Type Culture Collection (ATCC). All cell lines were authenticated with AuthentiFiler PCR Amplification Kit (Applied Biosystems) and routinely tested for mycoplasma infection. All cell lines were cultured in Dulbecco's modified Eagle's medium (DMEM)-high glucose media supplemented with 10% fetal bovine serum (FBS) and 1% penicillin-streptomycin (PS) (Gibco). For in vitro T cell culturing, RPMI 1640 media was used with 10% FBS and 1% PS supplemented with 25 mM HEPES buffer (Gibco), 50 μM 2-mercaptoethanol (SigmaAldrich) and 1 ng/mL IL-2 (R&D Systems). Cells were cultured in a 37° C. humidified incubator supplied with 5% CO₂. Multiple stable knockdown HCC cell lines were established using lentiviral approach as previously described (58, 59). shRNAs targeting IFNGR1, IFNGR2, JAK1, JAK2, STAT1, IRF1, Nampt or nontargeting control (NTC) were inserted into pLKO.1-puro vector and transfected into Hep3B or Hepa1-6 cells, which were then selected by puromycin. shRNA sequences were provided in table 1. HCC cell lines were treated with IFN-γ for 24 or 48 hours as indicated.

TABLE 1
shRNA/sgRNA target sequences

| | Target Sequence |
|---|---|
| shIFNGR1-1 | GCCTGCATCAATATTTCTCAT |
| | (SEQ ID NO: 1) |
| shIFNGR1-2 | GTTTCAGCAGAAGGAGTCTTA |
| | (SEQ ID NO: 2) |
| shIFNGR2-1 | GGAGGAATCCAACAGGTCAAA |
| | (SEQ ID NO: 3) |
| shIFNGR2-2 | GTCGGGCATTTAAGCAACATA |
| | (SEQ ID NO: 4) |
| shJAK1-1 | GCATGGAACCAACGACAATGA |
| | (SEQ ID NO: 5) |
| shJAK1-2 | GAAAGACAAGACGCTGATTGA |
| | (SEQ ID NO: 6) |
| shJAK2-1 | GCAAAGATCCAAGACTATCAT |
| | (SEQ ID NO: 7) |
| shJAK2-2 | GGGCAGAATTAGCAAACCTTA |
| | (SEQ ID NO: 8) |
| shSTAT1-1 | GAACAGAAATACACCTACGAA |
| | (SEQ ID NO: 9) |
| shSTAT1-2 | CGACAGTATGATGAACACAGT |
| | (SEQ ID NO: 10) |
| shIRF1-1 | CCTCTGTCTATGGAGACTTTA |
| | (SEQ ID NO: 11) |
| shIRF1-2 | GCGTGTCTTCACAGATCTGAA |
| | (SEQ ID NO: 12) |
| shNampt-1 | CCACCTTATCTTAGAGTCATT |
| | (SEQ ID NO: 13) |
| shNampt-2 | TCAGCGATAGCTATGACATTT |
| | (SEQ ID NO: 14) |
| Nampt^{KO} | CACCGTGTACGAGTCGGTGGC |
| | CAGC |
| | (SEQ ID NO: 15) and |
| | CACCGAGTAAGGAAGGTGAAA |
| | TACG |
| | (SEQ ID NO: 16) |

RNA extraction, reverse transcription, qPCR. Total RNA was extracted from frozen mouse HCC tissues, HCC cell lines, or primary T cells with TRIzol reagent (Ambion by Life Technologies). cDNA was prepared by reverse transcription using the GeneAmp RNA PCR Core Kit (Applied Biosystems). Quantitative real-time polymerase chain reaction (qRT-PCR) amplifications of NAMPT and internal control 18S were performed using SYBR Green PCR Master Mix (Applied Biosystems) with StepOne Real-Time PCR System (Applied Biosystems). Primers were listed in table 2.

TABLE 2
Primer sequences

| Primer | Sequence |
|---|---|
| Human NAMPT | Forward: |
| | CAGCAGCAGAACACAGTACCA |
| | (SEQ ID NO: 17) |
| | Reverse: |
| | CTGACCACAGATACAGGCACT |
| | (SEQ ID NO: 18) |
| Human 18S | Forward: |
| | GAGGATGAGGTGGAACGTGT |
| | (SEQ ID NO: 19) |
| | Reverse: |
| | AGAAGTGACGCAGCCCTCTA |
| | (SEQ ID NO: 20) |
| Human NAMPT | Forward: |
| | GTGTCGCACCCTGTCAAAG |
| | (SEQ ID NO: 21) |
| (STAT1-953 ChIP) | Reverse: |
| | GGGGACAAGACCTAATTGAACC |
| | (SEQ ID NO: 22) |
| Human NAMPT | Forward: |
| | GCAACCACGCATGAGAACTG |
| | (SEQ ID NO: 23) |
| (STAT1-1106 ChIP) | Reverse: |
| | CTTTGACAGGGTGCGACAC |
| | (SEQ ID NO: 24) |
| Mouse Nampt | Forward: |
| | GGGGCATCTGCTCATTTGGT |
| | (SEQ ID NO: 25) |
| | Reverse: |
| | AAGCCGTTATGGTACTGTGCT |
| | (SEQ ID NO: 26) |
| Mouse 18s | Forward: |
| | ACATCGACCTCACCAAGAGG |
| | (SEQ ID NO: 27) |
| | Reverse: |
| | TCCCATCCTTCACATCCTTC |
| | (SEQ ID NO: 28) |

Chromatin immunoprecipitation (ChIP). Hep3B cells were treated with 50 ng/ml IFN-γ for 24 hours prior to the experiment. DNA crosslinking was achieved by 3.7% formaldehyde fixation. Upon cell lysis with SDS buffer, sonication was performed to shear DNA into fragments, which were then incubated with antibodies against STAT1 or immunoglobulin G (IgG) control (table S3) in the presence of Protein A Agarose/Salmon Sperm DNA Beads (Merck Millipore) overnight. DNA-protein-antibody complexes were then washed with salt buffers with gradient concentrations and eluted in 1% SDS/NaHCO₃. ChIP DNA samples were analyzed by qRT-PCR using primers targeting the putative GAS elements respectively (table 2).

Sample preparation for extracellular NAD detection. For serum or cultured medium metabolite extraction, 100 μL of the sample was mixed with 400 μL of chilled pure methanol, followed by 30 seconds of mixing and incubation at −80° C. for 15 minutes. The mixtures were centrifuged at 4° C. for 15 minutes at 13,500 rpm, with the supernatants being transferred to new tubes and dried using a vacuum centrifuge.

LC-MS/MS Analysis. The supernatants obtained from the sample preparation procedures were subjected to LC-MS/MS analysis using an LCMSX500R (AB SCIEX). A standard mobile phase was employed, with phase A consisting of water containing 10 mM ammonium acetate, and phase B consisting of acetonitrile. The flow rate was maintained at 0.3 mL/min, and the sample injection volume was set at 10 μL. The column temperature was controlled at 37° C. Analyte separation was achieved using a hypercarbon column (5 μm particles, 2.1 mm×150 mm, Thermo). The LC solvent gradient followed this profile: from 0 to 5 minutes, phase B was maintained at 15%; from 5 to 15 minutes, phase B was linearly increased from 15% to 95%; from 15 to 15.1 minutes, phase B was linearly decreased from 95% to 15%; and from 15.1 to 20 minutes, phase B was maintained at 15%.

The electrospray ionization source was operated in the positive mode with the following settings: ion source gas 1 at 80 psi, ion source gas 2 at 80 psi, curtain gas at 30 psi, temperature at 550° C., ionspray voltage floating at +5500 V, delustering potential at 100 V, and collision energy at 10 eV. Multiple reaction monitoring experiments were selected for MS acquisition. Ion transitions from precursor to product ions were used for peak identification. Data analysis, including peak identification and integration, was performed using Analyst® Software (AB SCIEX). NAD was identified based on exact mass and retention time matched to commercial standards.

Cell surface and intracellular staining for flow cytometry. Cell suspensions from tumor tissues were stained with LIVE/DEAD Fixable Violet Dead Cell Stain Kit (Invitrogen) prior to cell surface staining. For cell surface staining, cells were incubated with fluorochrome conjugated primary antibodies at 4° C. for 30 minutes in the dark. Intracellular staining was performed using the eBioscience Foxp3/Transcription Factor Staining Buffer Set (Invitrogen). Cells were analyzed with the analyzer BD LSRFortessa (BD Biosciences). Primary antibodies used and the dilution were indicated in table 3. Software FlowJo was used for data analysis.

TABLE 3
Antibodies used

| Antibody | Application | Clone | Dilution/Amt used | Cat no. | Company |
|---|---|---|---|---|---|
| Human STAT1 | ChIP | / | 2 μg | ab234400 | Abcam |
| IgG control | ChIP | / | 2 μg | 10500C | Invitrogen |
| Mouse CD16/32 | FC | 93 | 1:100 | 101301 | Biolegend |
| Mouse CD45 | FC | 30-F11 | 1:100 | 103127 | Biolegend |
| Mouse CD8b | FC | YTS156.7.7 | 1:100 | 126619 | Biolegend |
| Mouse CD44 | FC | IM7 | 1:100 | 103005 | Biolegend |
| Mouse CD62L | FC | MEL-14 | 1:100 | 104431 | Biolegend |
| Mouse PD-1 | FC | 29F.1A12 | 1:50 | 135219 | Biolegend |
| Mouse TIM-3 | FC | RMT3-23 | 1:100 | 119705 | Biolegend |
| Mouse CD3 | FC | 17A2 | 1:100 | 100205 | Biolegend |
| Mouse CD4 | FC | GK1.5 | 1:100 | 100429 | Biolegend |
| Mouse FOXP3 | FC | FJK-16s | 1:100 | 45-5773-82 | Invitrogen |
| Mouse CD8a | IHC | D4W2Z | 1:200 | 98941 | Cell Signaling |
| Mouse CD4 | IHC | EPR19514 | 1:1000 | ab183685 | Abcam |
| Mouse CD161 | IHC | E6Y9G | 1:200 | 38197 | Cell Signaling |

ChIP: chromatin immunoprecipitation;
FC: flow cytometry;
IHC: immunohistochemistry

In vitro T cell assays. Splenic T cells from 5- to 7-week-old C57BL/6N male mice were isolated with Mouse T Cell Enrichment Columns (R&D Systems) following the manufacturer's instruction. Isolated T cells were cultured in the presence of Dynabeads Mouse T-Activator CD3/CD28 (Gibco). T cells were treated with NAD at indicated concentration in the presence or absence of 1 μM A438079 (P2X7R antagonist; MedChemExpress). T cells were cultured for 72 hours and analyzed by flow cytometry. Cell death of T cell was measured using MEBCYTO Apoptosis Kit (MBL). Treg proportion was defined by CD4⁺FOXP3⁺. T cell exhaustion was evaluated by PD-1 expression. T cells were also harvested for transcriptome sequencing. For co-culture experiment with Hepa1-6 cells, isolated T cells were first labeled with CellTrace Violet (CTV; Invitrogen) and cultured with Heap1-6 cells (EV or shNampt) pre-treated with IFN-γ in 1:1 ratio for 72 hours and CTV signals were analyzed by flow cytometry.

Animal studies. Animal procedures performed in this study were approved by the Committee on the Use of Live Animals in Teaching and Research of The University of Hong Kong and adhered to the Animals (Control of Experiments) Ordinance of Hong Kong.

To study the sensitizing effect of NAMPT deficiency to anti-PD-1 treatment, subcutaneous implantation and hydrodynamic tail vein injection (HDTVi) HCC models were utilized. For subcutaneous tumor model, 2×10⁶ Hepa1-6 cells (EV or shNampt) were resuspend in 50% Matrigel in 1×PBS (BD Biosciences) and implanted subcutaneously into both flanks of 5- to 7-week-old C57BL/J male mice. Treatment of anti-PD-1 monoclonal antibody (clone RMP1.14; Bio X Cell) began 7 days after implantation when tumors were palpable. Mice were treated with anti-PD-1 (250 μg per mouse) twice a week intraperitoneally. Tumor sizes were measured using calipers every other day. Tumors were harvested on day 27 for tumor size and immune cell analysis. Tumors were dissociated and prepared for cell staining as described previously (58, 59). For HDTVi HCC model, a CRISPR-cas9 based vector carrying sgRNA targeting Trp53 and Nampt simultaneously was generated and transposon system was used to overexpress c-myc vector. These plasmids were diluted in saline and a volume of 10% of the mouse body weight plasmid mixture was injected into lateral tail vein of 8-to 10-week-old male C57BL/6N mice within 6-8 seconds. sgRNA sequences were listed in table 1. Anti-PD-1 treatment started 21 days after HDTVi and treated as described above. Mice were sacrificed upon reaching humane endpoint or at the end of the experiment.

To study the therapeutic effect of P2X7R antagonist, orthotopic implantation and HDTVi HCC models were utilized. For orthotopic implantation model, 3×10⁶ Hepa1-6 cells were resuspend in 100% Matrigel and implanted into left liver lobe of 5- to 7-week-old C57BL/J male mice. Treatment of A438079 (P2X7R antagonist) began 3 days after implantation. Mice were treated with A438079 (30 mg/kg) every other day intraperitoneally. Tumors were harvested on day 16 for tumor size and tissue sample collection. For HDTVi HCC model, vector carrying Trp53-targeting sgRNA was injected with transposon system vector with c-myc into C57BL/6N mice as described above. A438079 treatment began on day 14 while anti-PD-1 and CD40 agonist therapies started on day 21. Regimens of A438079 and anti-PD-1 were as above. CD40 agonist (clone FGK4.5, Bio X Cell) was treated at a dose of 125 ug per mouse weekly via intraperitoneal injection. Mice were sacrificed upon reaching humane endpoint or at the end of the experiment.

Immunohistochemical staining. Formalin-fixed paraffin-embedded (FFPE) mouse HCC tissue sections were stained with primary antibody targeting CD8⁺ T cells, CD4⁺ T cells and NK cells respectively at 4° C. overnight after antigen retrieval treatment by boiling the sections in either 1 mM EDTA buffer (pH 7.8) or 10 mM Tris and 1 mM EDTA buffer (pH 9.0) for 15 minutes. Sections were incubated with horseradish peroxidase-conjugated secondary antibody (Dako) at room temperature for 30 minutes the next day followed by development with 3,3′-diaminobenzidine (Sigma-Aldrich). Primary antibodies used were indicated in table S3.

Bulk RNA sequencing. Bulk RNA sequencing was performed on 3 human HCC cell lines (Hep3B, Huh7 and HepG2) treated with vehicle or 50 ng/mL IFN-γ for 24 hours. Transcriptome sequencing was also performed on mouse splenic T cells treated with 10 μM NAD or vehicle for 72 hours. The library preparation and sequencing were carried out by Novogene (Beijing, China).

Statistical analysis. Data were analyzed by Student's t test unless otherwise specified and were expressed as means±SD. P values<0.05 were considered statistically significant. GraphPad Prism 8.0 or above software (GraphPad Software Inc.) is used for data analysis.

### Results

### NAMPT Conferred Resistance to Anti-PD-1 and Was Regulated by IFN-γ/JAK/STAT Pathway

As discussed above, IFN-γ pathway has been identified as a contributor to ICI resistance. To discover ICI resistant mechanisms in HCC, HCC cell lines were treated with IFN-γ and performed transcriptomic analysis. Among the three human HCC cell lines that were used, 208 genes were commonly upregulated. GO term enrichment analysis showed that NAD biosynthetic process was one of the most enriched pathways among the other well-known IFN-γ-induced pathways (FIG. 1A-1C). The upregulation of NAMPT, the key enzyme in NAD salvage pathway, was confirmed in all three HCC cell lines (FIG. 1D-1F). To further explore the association of NAMPT and ICI resistance, NAMPT expression was studied in two anti-PD-1 resistant mouse HCC models and NAMPT was upregulated in tumors treated with anti-PD-1 (FIG. 1G-1H). CIBERSORTX analysis of human HCC TCGA database revealed that NAMPT expression level in HCC patients was negatively correlated with CD8 T cells and activated NK cells (FIG. 1I-1J), reflecting an immunosuppressive role of NAMPT. An online database of spatial transcriptomic (ST) analysis of HCC samples (SpatialTME) was also assessed. It included ST data from HCC samples of anti-PD-1 responders (R) and non-responders (NR)(34). In both R and NR, it was observed that NAMPT expression was highly expressed in tumor region and overlapped with IFNG signature (IFNGR1, IFNGR2, JAK1, JAK2, STAT1, IRF1) hinting the regulation of NAMPT by IFN-γ. Moreover, the CD8A and KLRK1 expression patterns, representing CD8⁺ T cells and NK cells respectively, were mutually exclusive with NAMPT expression, in consistent with negative correlation of NAMPT and CD8 T cells or activated NK cells in the CIBERSORTX analysis. A higher expression of NAMPT was observed in NR accompanied by lower expressions of CD8A and KLRK1 (FIG. 1M).

As JAK/STAT pathway is the canonical IFN-γ signaling pathway, it was investigated if NAMPT was also regulated by this pathway. Key components of this pathway were knocked down including IFNGR1, IFNGR2, JAK1, JAK2, STAT1 and IRF1 in human HCC cell line, Hep3B, and it was shown that the deficiency of each component could reverse the induction of NAMPT upon IFN-γ treatment (FIG. 2A-2F). IFN-γ binds to IFNGR-1/IFNGR-2 to mediate the phosphorylation and activation of JAKs which in turn activates the phosphorylation of STAT1, forming homodimer to translocate into nucleus to bind to genes encompassing consensus sequence GAS, 5′-TTCN₂₋₄GAA-3′, to initiate its transcription as a primary response (35). Putative GAS sequences were identified at NAMPT promoter region. Thus, it was hypothesized that STAT1 dimer could promote NAMPT transcription via binding to GAS of NAMPT directly. Chromatin immunoprecipitation (ChIP) was performed with STAT1 antibody and qPCR results demonstrated that there were enriched binding of STAT1 at NAMPT promoter region upon IFN-γ exposure (FIG. 2G-2I).

### Extracellular NAD Skewed T Cells to Immunosuppressive Phenotypes

To understand the role of NAMPT and NAD metabolism in shaping the TME, stable Nampt knockdown clones were established (shNampt as compared to EV) in mouse HCC cell line, Hepa1-6. It was observed that Nampt was dramatically induced by IFN-γ and was rescued upon Nampt knockdown (FIG. 3A). Co-culture experiments were performed with mouse splenic T cells and the Nampt-deficient HCC cells. The results demonstrated that HCC cells inhibited T cell proliferation, and this is further suppressed when the HCC cells were pretreated with IFN-γ. Interestingly, this effect could be abrogated by Nampt knockdown in HCC cells (FIG. 3B-3C). As Nampt is a key enzyme regulating NAD metabolism and extracellular NAD suppress T cells, it was hypothesized if the rescuing effect on T cell proliferation is due to the reduction of extracellular NAD level upon Nampt knockdown in HCC cells. Conditioned media was harvested from Nampt knockdown HCC cells treated with IFN-γ. Indeed, NAD level in conditioned medium was elevated upon IFN-γ treatment and it was abrogated by Nampt knockdown (FIG. 3D), suggesting that extracellular NAD can contribute to the IFN-γ-mediated T cell suppression.

Therefore, the role of extracellular NAD in T cell inhibition was studied. P2X7R is a known NAD receptor, thus mouse splenic T cells were treated with NAD in the presence or absence of P2X7R antagonist (A438079). The results showed that NAD induced both CD4⁺ and CD8⁺ T cell apoptosis and this could be rescued by the addition of P2X7R antagonist (FIG. 3E-3G). Moreover, NAD promoted T cell differentiation into Treg and this could be abrogated by P2X7R antagonist (FIG. 3H-3L). NAD also induced PD-1 expression on both CD4⁺ and CD8⁺ T cells in a dose-dependent manner (FIG. 3M-3R). To further explore how NAD affects T cells, bulk RNA sequencing was performed on NAD-treated T cells. Consistent with the flow cytometry analysis, it was observed that NAD upregulated multiple Treg-related genes (Csf2, Il2, Il10, Ccr4, Ccl22, Foxp3, Il2ra and Tnfrsf18) and exhaustion markers (Pdcd1 and Haver2) while downregulated cytotoxic markers like Gzma and Gzmk (FIG. 3S). GSEA analysis revealed that IL-2/STAT5 and TGF-beta signaling were positively enriched in NAD-treated T cells (FIG. 3T-3U). These two pathways were both important in regulating Treg population (36). Together, these indicated that NAD suppressed T cell functions while skewed them to immunosuppressive phenotypes.

### Nampt Deficiency Sensitized HCC to Anti-PD-1 Treatment

The results presented herein demonstrated that knockdown of Nampt in HCC cells reduced extracellular NAD level, which suppressed T cells. Therefore, it was hypothesized if Nampt deficiency could sensitize HCC to anti-PD-1 treatment by relieving T cell suppression. Nampt knockdown clone (sh2) or EV Hepa1-6 that were established were subcutaneously injected the into immune-competent mice and treated these mice with anti-PD-1 (FIG. 4A). While anti-PD-1 reduced tumor size of EV Hepa1-6 tumors, it was observed that anti-PD-1 could further suppressed tumor growth upon Nampt knockdown (FIG. 4B-4G). It was studied how intratumoral T cells were affected. Anti-PD-1 induced CD8⁺ T cell infiltration in both EV and shNampt tumors (FIG. 4H-4K). Anti-PD-1 also induced exhaustion marker expression (PD-1 and TIM-3) on CD8⁺ T cells but the expression levels were lower in shNampt tumors (FIG. 4L-4N). Similarly, anti-PD-1 increased CD4⁺ T cells in both EV and shNampt tumors (FIG. 4O). Moreover, anti-PD-1 elevated exhaustion marker expression on CD4⁺ T cells but to a lesser extent in shNampt tumors (FIG. 4P-4R). This showed that T cells were less exhausted in Nampt deficient tumors. The effect of Nampt deficiency was further investigated in an anti-PD-1 resistant murine HCC model that was previously established (37). In this model, it was demonstrated that while anti-PD-1 treatment could not improve the survival outcomes of mice harboring the Trp53 knockout and c-myc overexpression, simultaneously knockout of Nampt could significantly extend the survival of mice. More importantly, Nampt deficiency further prolonged HCC-bearing mice survival upon anti-PD-1 treatment (FIG. 4S-4T). At the endpoint of the experiment, it was also observed that Nampt deficiency reduced tumor incidence (7 out of 11), and this could be further improved by anti-PD-1 treatment (2 out of 11) (FIG. 4U). Together, these data illustrated that Nampt deficiency sensitized HCC tumors to anti-PD-1 treatment.

### P2X7R Antagonist Suppressed Tumor Growth and Worked Synergistically With Immunotherapies

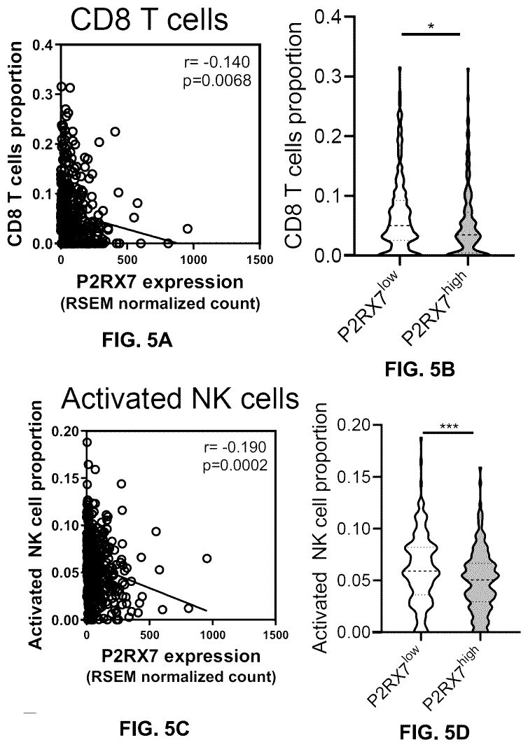

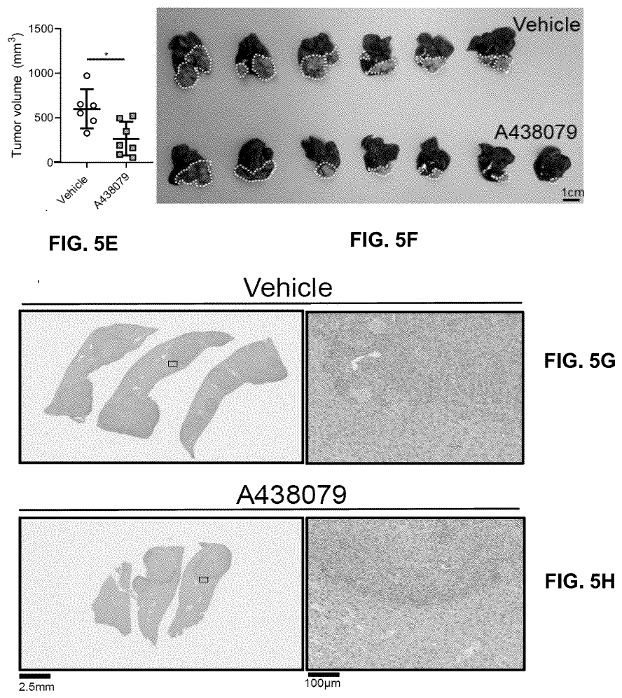

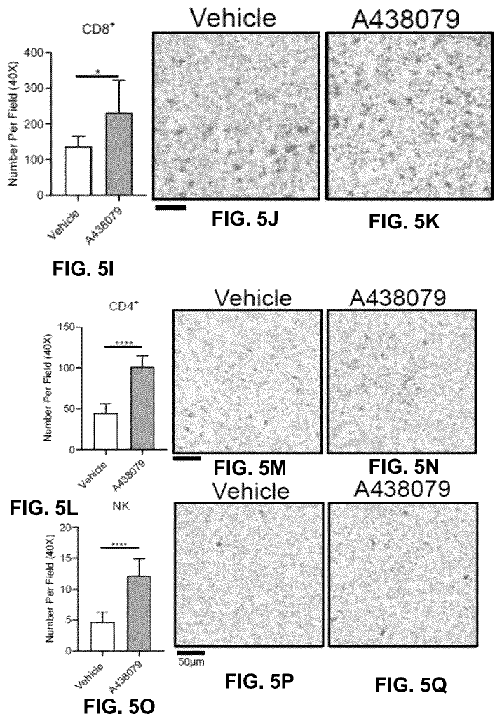

The data presented herein showed that the immunosuppressive effect of NAD could be rescued by blocking P2X7R in vitro. Interestingly, CIBERSORTX analysis showed that P2X7R expression level in HCC patients was also negatively correlated with CD8 T cells and activated NK cells (FIG. 5A-5D), suggesting a role of NAD sensing in ICI resistance. The therapeutic value of P2X7R antagonist was explored. It was demonstrated that P2X7R antagonist (A438079) suppressed tumor growth in an orthotopic implantation tumor model (FIG. 5E-5F). It was observed that tumors were more encapsulated upon antagonist treatment while those from vehicle group had an irregular growth front (FIG. 5G-5H). The shrinkage of tumor size was accompanied by increased infiltration of CD8⁺, CD4⁺ T cells and NK cells (FIG. 5I-5Q).

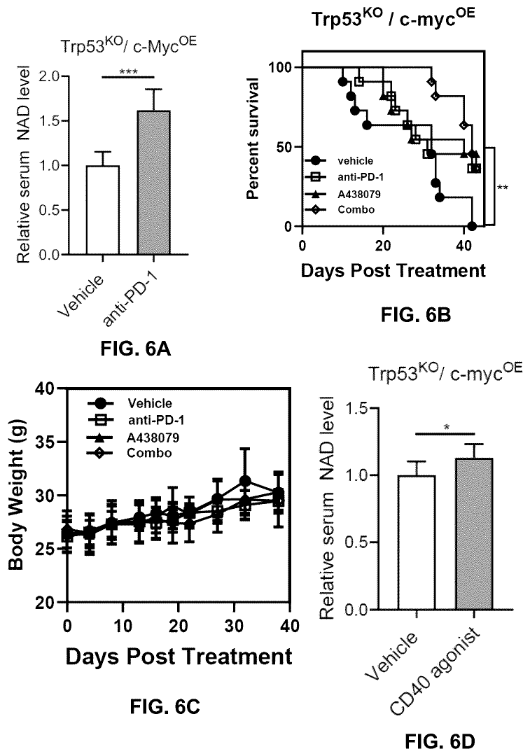

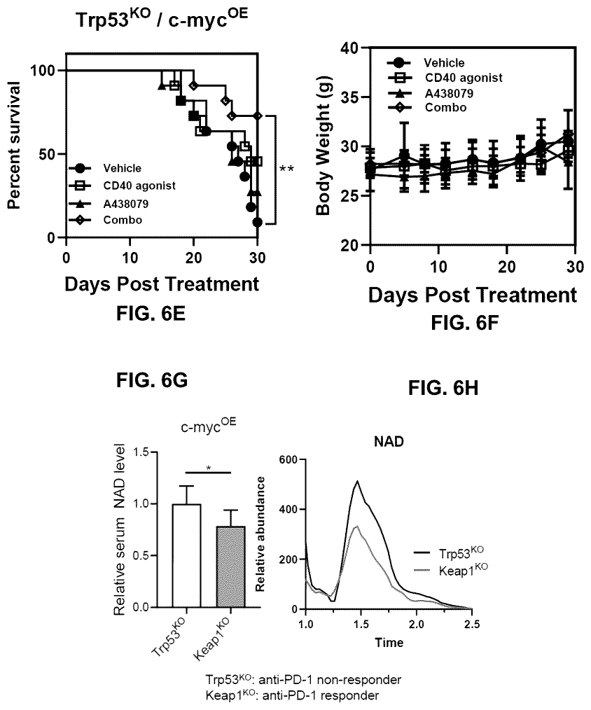

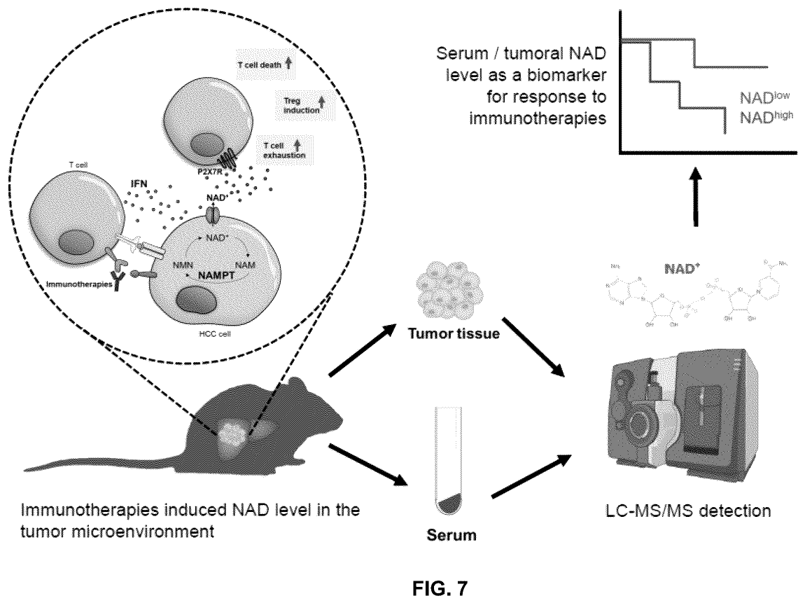

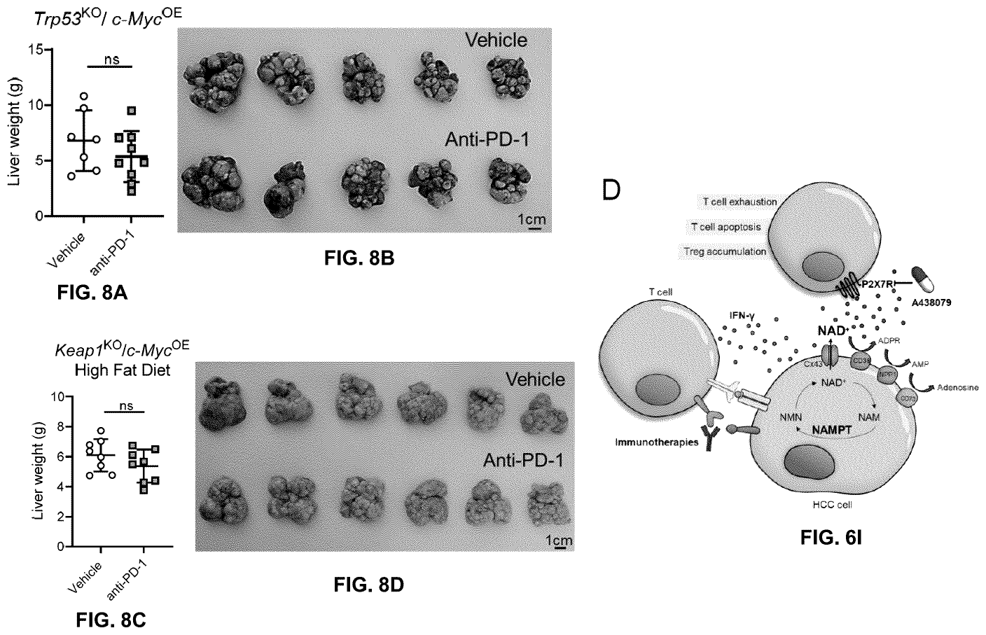

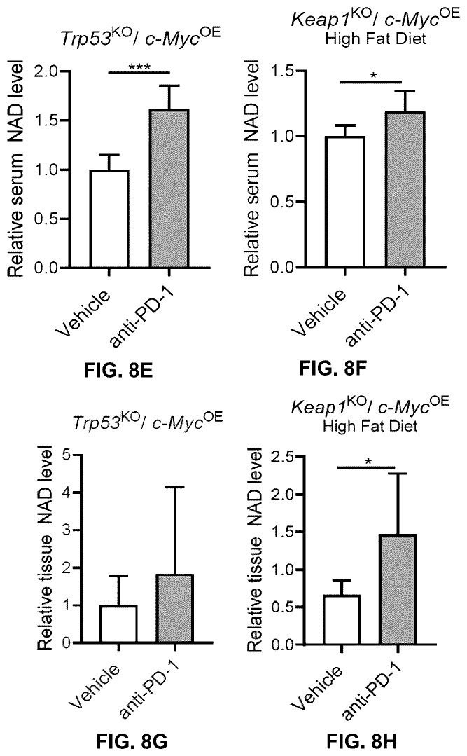

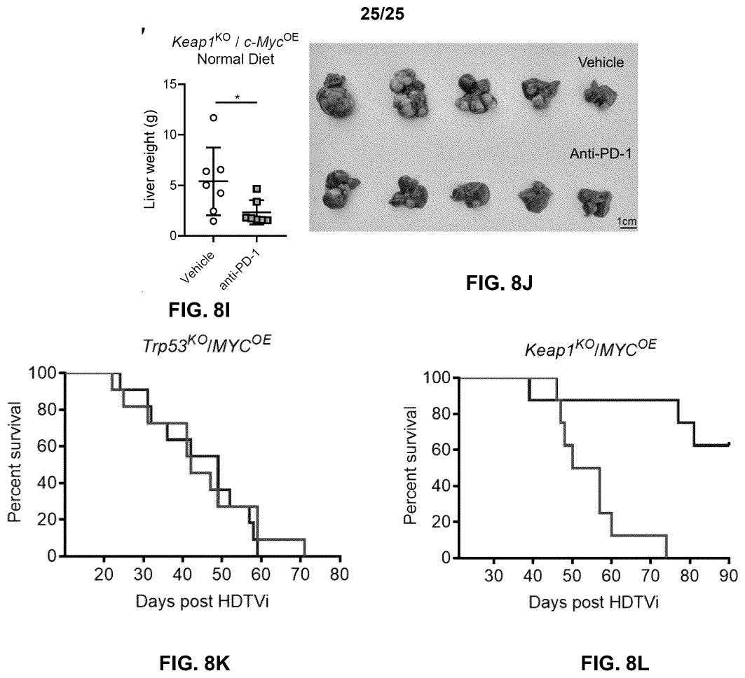

Whether P2X7R antagonist could be applied in combination with IFN-γ inducing immunotherapies was further investigated. It was observed that IFN-γ inducing immunotherapies elevated serum NAD level, which can contribute to immunosuppression. It was demonstrated that P2X7R antagonist prolonged HCC-bearing mice survival in combination with either anti-PD-1 or CD40 agonist treatment (FIG. 6A-6F). To explore if serum NAD level could be a predictive marker to ICI responses, serum NAD levels of anti-PD-1 responsive and resistant murine HCC tumor models were compared and demonstrated that serum NAD level was elevated from anti-PD-1 resistant model (FIG. 6G-6H). Overall, the data presented herein revealed that IFN-γ induced extracellular NAD via upregulation of NAMPT. NAD suppressed T cells and contributed to ICI resistance. The blockade of NAD sensing receptor P2X7R can be a therapeutic target to overcome ICI resistance (FIG. 6I).

### Discussion

Combination treatment becomes a valuable therapeutic approach in order to improve the clinical outcome of ICIs. Overcoming the resistant mechanisms of ICIs is an attractive option. It was identified NAMPT as a contributor to ICI resistance based on multiple analyses. Firstly, ICI treatment induced IFN-γ in the TME and it was demonstrated that NAMPT was induced by ICI treatment or IFN-γ and regulated by the JAK/STAT pathway. Previous study also identified NAMPT as a downstream target of STAT1 in macrophages (38). Secondly, it was observed that NAMPT expression in HCC patients was negatively correlated with CD8⁺ T cells and NK cells infiltration. NAMPT is involved in the immune regulation through two ways: [1] NAD metabolism and [2] extracellular NAMPT. NAMPT regulates the availability of NAD and NAD is important to the proper functioning of multiple metabolic and epigenetic enzymes. In a recent study in HCC, it was demonstrated that NAMPT was involved in the NAD-dependent α-KG production and this promoted PD-L1 expression on cancer cells via TETI regulation (26). NAMPT can also be secreted to the surroundings and the extracellular NAMPT (eNAMPT) has pleiotropic effects on myeloid cells (39). Interestingly, there is a recent study showing that eNAMPT promoted the expansion of MDSCs in tumors and targeting NAMPT could reduce MDSCs while increased IFN-γ producing CD8⁺ T cells in the tumors (40). Here, it was revealed that NAMPT could also mediate its immunosuppressive role through extracellular NAD production. It was shown that NAMPT deficiency in HCC cells decreased extracellular NAD and the tumor infiltrating T cells were less exhausted. Importantly, NAMPT suppression could sensitize HCC tumors to anti-PD-1 treatment. NAD was an immunosuppressive metabolite and NAMPT regulated the availability of NAD, pointing to the third way that NAMPT mediates its immunomodulatory role.

It has been observed that NAD induced T cell apoptosis through activating P2X7R by ART2 mediated ADP-ribosylation. NAD was utilized by ART2 to ADP-ribosylate P2X7R, resulting in calcium influx, cell membrane pore formation and phosphatidylserine exposure (28). In the context of lung cancer, ART1 was highly expressed and promoted NAD-induced cell death of tumor infiltrating CD8⁺ T cells, which could be reversed by ART1 blockade (29). Apart from apoptosis, it was demonstrated that the extracellular NAD induced T cell exhaustion and promoted Treg accumulation. These effects were mediated by the receptor P2X7R. Therefore, P2X7R antagonist serves as a therapeutic option. Indeed, the data presented herein demonstrated that P2X7R antagonist suppressed tumor growth and increased tumor infiltration of T cells and NK cells. It also worked synergistically with immunotherapies to improve survival outcomes in mice HCC model. Admittedly, P2X7R is also the receptor for ATP, serving as a danger signal to promote inflammatory response. It has been reported that ATP bound to P2X7R and activated NLRP3 inflammasome, inducing the production of pro-inflammatory cytokine IL-1β by DCs, enhancing anti-tumor responses (41). However, in the TME, ATP is rapidly converted to AMP and adenosine, two immunosuppressive metabolites, by the highly abundant ectoenzymes CD39 and CD73, as previously shown (42). Therefore, the influence of P2X7R blockade on ATP signaling is greatly limited by the ATP availability in the TME. Apart from P2X7R blockade, it can be explored that the ART responsible for the NAD-induced P2X7R ribosylation in HCC which could also be a therapeutic target.

Patients' responses to ICIs in HCC are heterogenous. Predictive markers for ICI responses have been hotly pursued in the field because it enables the identification of patients that are most suitable for ICIs. Currently, the most commonly used or studied markers across different cancer types include tumor mutation burden (TMB), tumoral PD-L1 expression and CD8⁺ T cell infiltration. However, it is shown that these markers can only be predictive in some cancer types and their performance varied across studies. Some studies demonstrated that high TMB was associated with better response to anti-PD-1/anti-PD-L1 treatment in multiple cancer types, but HCC patients were not included (43, 44). On the other hand, pan-tumor studies with HCC patients showed that TMB failed to predict anti-PD-1/anti-PD-L1 responses in some cancer types and its predictive ability was dependent on immune cell or CD8⁺ T cell infiltration (45, 46). Moreover, it was showed that only a small percentage of HCC patients (2.1%) was considered TMBhigh (46) while the response rate of HCC patients to anti-PD-1 treatment was around 20% (4, 5), suggesting that there are certain proportion of TMB^{low} HCC patients responds to anti-PD-1 therapies. As the target of anti-PD-1/anti-PD-L1 therapy, tumoral PD-L1 expression has been used as a diagnostic marker for anti-PD-1 treatment (47). However, the predictive ability of PD-L1 to anti-PD-1 response varied across different studies (48, 49). This could be attributed to technical variations (including antibody clones, types of tissues, PD-L1 positivity threshold) and biological features of PD-L1 (50,51). PD-L1 expression is regulated at multiple levels. At the transcript level, PD-L1 could be induced by interferon and hypoxia (52). At the protein level, it was previously shown that CMTM4 and CMTM6 directly regulated PD-L1 expression on cancer cell surface (53-55). Therefore, PD-L1 expression is highly heterogeneous in different tumor regions even in the same patient. CD8⁺ T cells being the targeted population of ICIs, its presence in the tumor regions is a reasonable predictive marker. It was demonstrated that patients with higher number of CD8⁺ T cells in tumor prior to treatment responded better to anti-PD-1 treatment in melanoma patients (56, 57). However, similar to PD-L1 expression, CD8⁺ T cell infiltration also varies in different tumor regions and thus its predictive ability will be affected by the regions of biopsies. Moreover, tumor biopsies involving invasive procedures are often needed to perform assessment on these markers. Therefore, biomarkers from peripheral blood, which can be easily obtained routinely, will be more ideal to serve as a predictor to ICIs and proposed scrum NAD level as a predictive biomarker. Murine HCC models that are responsive and resistant to anti-PD-1 treatment was previously established (37). It was shown that HCC with genotype Trp53^{KO}/c-myc^{OE} had low CD8⁺ T cell infiltration and was resistant to anti-PD-1 treatment while HCC with genotype Keap1^{KO}/c-myc^{OE} had high CD8⁺ T cell infiltration and responded to anti-PD-1 treatment. Importantly, it was demonstrated that serum NAD level prior to treatment was higher in resistant HCC model (Trp53^{KO}/c-myc^{OE}) than the responsive one (Keap1^{KO}/c-myc^{OE}). Moreover, immunotherapies treatment that increased IFN-γ level in the TME also induced serum NAD level in tumor bearing mice and the combination use of P2X7R antagonist with immunotherapies improved survival outcomes. These indicated that serum NAD level has predictive value to anti-PD-1 responses.

Overall, the current study revealed a new ICI resistance mechanism mediated by NAMPT. ICIs induce IFN-γ production and upregulated NAMPT, which contributed to elevated extracellular NAD level. NAMPT deficiency in HCC sensitized HCC to anti-PD-1 treatment. NAD suppressed T cell functions and promoted T cell differentiation to Treg. The blockade of P2X7R reversed the immunosuppressive effect of NAD and worked with immunotherapies to improve treatment efficacy, serving as a combination therapeutic approach for ICI-resistant tumors. Importantly, serum NAD level can be predictive biomarker for ICI therapies.

As ICIs emerge to be valuable treatment options in multiple cancer types, the mechanism that mediates immune evasion upon ICI treatment has been the key focus of exploration. ICIs enhanced anti-tumor responses via inducing IFN-γ secretion by T cells (8). However, despite the important role of IFN-γ in T cell-mediated cancer cell killing, multiple studies across different cancer types convergently pointed to that IFN-γ pathway also contribute to ICI resistance. A recent in vivo CRISPR screen on cancer models treated with ICIs identified IFN sensing pathway as key immune evasion mediator (9). Loss of IFN signaling sensitized tumors to ICIs. Prolonged exposure to IFN-γ altered the epigenome and transcriptome in cancer cells, conferring them the resistance to ICIs via different mechanisms (10, 11). IFN-γ is known to be an inducer of PD-L1 expression on tumor cells, contributing to immune evasion via interaction with PD-1 on T cells (12-14). IFN-γ also upregulated indoleamine-2,3-dioxygenase (IDO), which converts an essential amino acid tryptophan to an immunosuppressive metabolite kynurenine (15). The upregulation of IDO in tumor cells or other immune cell types depleted tryptophan in the tumor microenvironment (TME) and deprived tumor-infiltrating T cells from this essential amino acid, suppressing their proliferation via GCN2 activation (16). On the other hand, kynurenine is an endogenous ligand for the transcription factor aryl hydrocarbon receptor (AHR)(17). It is demonstrated AHR-expressing dendritic cells (DC) promoted T cell differentiation to regulatory T cells (Treg) (18). Ablation of IDO in tumor cells reduced Treg recruitment in glioma model (19). Pre-clinical studies using IDO inhibitor together with ICIs showed synergistic effects in different tumor models and this led to their use in clinical trials (20-22).

The data presented herein revealed that ICI treatment elevated extracellular NAD level and conferred cancer cells resistance to ICIs. It was shown that NAMPT was upregulated by IFN-γ and ICIs leading to an increase of extracellular NAD level, which could be abrogated upon NAMPT knockdown. NAMPT deficiency in cancer cells sensitized tumors to anti-PD-1 treatment. Mechanistically, NAMPT was regulated by IFN-γ via JAK/STAT pathway. It was further demonstrated that extracellular NAD induced T cell apoptosis while promoted T cell differentiation to Tregs, which could be rescued by blockade of the P2X7R, the NAD receptor. P2X7R blockade worked in combination with immunotherapies. This study identified extracellular NAD as an immunosuppressive metabolite that enables immune escape of cancer cells and the P2X7R as a therapeutic target to improve ICI efficacy.

### REFERENCES

1. Llovet J M, Kelley R K, Villanueva A, Singal A G, Pikarsky E, Roayaie S, et al. Hepatocellular carcinoma. Nat Rev Dis Primers. 2021; 7(1):6.
2. Sangro B, Sarobe P, Hervas-Stubbs S, Melero I. Advances in immunotherapy for hepatocellular carcinoma. Nat Rev Gastroenterol Hepatol. 2021; 18(8):525-43.
3. Sharma P, Siddiqui B A, Anandhan S, Yadav S S, Subudhi S K, Gao J, et al. The Next Decade of Immune Checkpoint Therapy. Cancer Discov. 2021; 11(4):838-57.
4. El-Khoueiry A B, Sangro B, Yau T, Crocenzi T S, Kudo M, Hsu C, et al. Nivolumab in patients with advanced hepatocellular carcinoma (CheckMate 040): an open-label, non-comparative, phase ½ dose escalation and expansion trial. Lancet. 2017; 389(10088):2492-502.
5. Zhu A X, Finn R S, Edeline J, Cattan S, Ogasawara S, Palmer D, et al. Pembrolizumab in patients with advanced hepatocellular carcinoma previously treated with sorafenib (KEYNOTE-224): a non-randomised, open-label phase 2 trial. The Lancet Oncology. 2018; 19(7):940-52.
6. Yau T, Park J-W, Finn R S, Cheng A-L, Mathurin P, Edeline J, et al. Nivolumab versus sorafenib in advanced hepatocellular carcinoma (CheckMate 459): a randomised, multicentre, open-label, phase 3 trial. The Lancet Oncology. 2022; 23(1):77-90.
7. Finn R S, Qin S, Ikeda M, Galle P R, Ducreux M, Kim T Y, et al. Atezolizumab plus Bevacizumab in Unresectable Hepatocellular Carcinoma. N Engl J Med. 2020; 382(20):1894-905.
8. Gocher A M, Workman C J, Vignali D A A. Interferon-γ: teammate or opponent in the tumour microenvironment? Nature Reviews Immunology. 2022; 22(3):158-72.
9. Dubrot J, Du P P, Lane-Reticker S K, Kessler E A, Muscato A J, Mehta A, et al. In vivo CRISPR screens reveal the landscape of immune evasion pathways across cancer. Nature Immunology. 2022; 23(10):1495-506.
10. Benci J L, Xu B, Qiu Y, Wu T J, Dada H, Twyman-Saint Victor C, et al. Tumor interferon signaling regulates a multigenic resistance program to immune checkpoint blockade. Cell. 2016; 167(6):1540-54.
11. Qiu J, Xu B, Ye D, Ren D, Wang S, Benci J L, et al. Cancer cells resistant to immune checkpoint blockade acquire interferon-associated epigenetic memory to sustain T cell dysfunction. Nature Cancer. 2023:1-19.
12. Thiem A, Hesbacher S, Kneitz H, di Primio T, Heppt M V, Hermanns H M, et al. IFN-gamma-induced PD-L1 expression in melanoma depends on p53 expression. Journal of Experimental & Clinical Cancer Research. 2019; 38(1):1-15.
13. Abiko K, Matsumura N, Hamanishi J, Horikawa N, Murakami R, Yamaguchi K, et al. IFN-γ from lymphocytes induces PD-L1 expression and promotes progression of ovarian cancer. British journal of cancer. 2015; 112(9):1501-9.
14. Qian J, Wang C, Wang B, Yang J, Wang Y, Luo F, et al. The IFN-γ/PD-L1 axis between T cells and tumor microenvironment: hints for glioma anti-PD-1/PD-L1 therapy. Journal of neuroinflammation. 2018; 15(1):1-13.
15. Dai W, Gupta S L. Regulation of indoleamine 2,3-dioxygenase gene expression in human fibroblasts by interferon-gamma. Upstream control region discriminates between interferon-gamma and interferon-alpha. Journal of Biological Chemistry. 1990; 265(32):19871-7.
16. Munn D H, Sharma M D, Baban B, Harding H P, Zhang Y, Ron D, et al. GCN2 kinase in T cells mediates proliferative arrest and anergy induction in response to indoleamine 2,3-dioxygenase. Immunity. 2005; 22(5):633-42.
17. Opitz C A, Litzenburger U M, Sahm F, Ott M, Tritschler I, Trump S, et al. An endogenous tumour-promoting ligand of the human aryl hydrocarbon receptor. Nature. 2011; 478(7368):197-203.
18. Nguyen N T, Kimura A, Nakahama T, Chinen I, Masuda K, Nohara K, et al. Aryl hydrocarbon receptor negatively regulates dendritic cell immunogenicity via a kynurenine-dependent mechanism. Proceedings of the National Academy of Sciences. 2010; 107(46):19961-6.
19. Wainwright D A, Balyasnikova I V, Chang A L, Ahmed A U, Moon K-S, Auffinger B, et al. IDO Expression in Brain Tumors Increases the Recruitment of Regulatory T Cells and Negatively Impacts SurvivalIDO Regulates Treg Infiltration in Brain Tumors. Clinical cancer research. 2012; 18(22):6110-21.
20. Holmgaard R B, Zamarin D, Munn D H, Wolchok J D, Allison J P. Indoleamine 2,3-dioxygenase is a critical resistance mechanism in antitumor T cell immunotherapy targeting CTLA-4. Journal of Experimental Medicine. 2013; 210(7):1389-402.
21. Wainwright D A, Chang A L, Dey M, Balyasnikova I V, Kim C K, Tobias A, et al. Durable Therapeutic Efficacy Utilizing Combinatorial Blockade against IDO, CTLA-4, and PD-L1 in Mice with Brain TumorsIDO, CTLA-4, PD-L1 Synergistic Blockade in Brain Tumors. Clinical cancer research. 2014; 20(20):5290-301.
22. Fujiwara Y, Kato S, Nesline M K, Conroy J M, DePietro P, Pabla S, et al. Indoleamine 2,3-dioxygenase (IDO) inhibitors and cancer immunotherapy. Cancer Treatment Reviews. 2022:102461.
23. Katsyuba E, Romani M, Hofer D, Auwerx J. NAD+ homeostasis in health and disease. Nature metabolism. 2020; 2(1):9-31.
24. Demarest T G, Babbar M, Okur M N, Dan X, Croteau D L, Fakouri N B, et al. NAD+ metabolism in aging and cancer. Annual Review of Cancer Biology. 2019; 3:105-30.
25. Yaku K, Okabe K, Hikosaka K, Nakagawa T. NAD metabolism in cancer therapeutics. Frontiers in oncology. 2018; 8:622.
26. Lv H, Lv G, Chen C, Zong Q, Jiang G, Ye D, et al. NAD+ metabolism maintains inducible PD-L1 expression to drive tumor immune evasion. Cell metabolism. 2021; 33(1):110-27.
27. Bruzzone S, Guida L, Zocchi E, Franco L, Flora A D. Connexin 43 hemichannels mediate Ca2+-regulated transmembrane NAD+ fluxes in intact cells. The FASEB Journal. 2001; 15(1):10-2.
28. Seman M, Adriouch S, Scheuplein F, Krebs C, Freese D, Glowacki G, et al. NAD-induced T cell death: ADP-ribosylation of cell surface proteins by ART2 activates the cytolytic P2X7 purinoceptor. Immunity. 2003; 19(4):571-82.
29. Wennerberg E, Mukherjee S, Spada S, Hung C, Agrusa C J, Chen C, et al. Expression of the mono-ADP-ribosyltransferase ART1 by tumor cells mediates immune resistance in non-small cell lung cancer. Science translational medicine. 2022; 14(636):eabe8195.
30. Romagnani A, Rottoli E, Mazza E M C, Rezzonico-Jost T, De Ponte Conti B, Proietti M, et al. P2X7 receptor activity limits accumulation of T cells within tumors. Cancer Research. 2020; 80(18):3906-19.
31. Heske C M. Beyond energy metabolism: exploiting the additional roles of NAMPT for cancer therapy. Frontiers in oncology. 2020; 9:1514.
32. Garten A, Schuster S, Penke M, Gorski T, De Giorgis T, Kiess W. Physiological and pathophysiological roles of NAMPT and NAD metabolism. Nature Reviews Endocrinology. 2015; 11(9):535-46.
33. Sampath D, Zabka T S, Misner D L, O'Brien T, Dragovich P S. Inhibition of nicotinamide phosphoribosyltransferase (NAMPT) as a therapeutic strategy in cancer. Pharmacology & therapeutics. 2015; 151:16-31.
34. Liu Y, Xun Z, Ma K, Liang S, Li X, Zhou S, et al. Identification of a tumour immune barrier in the HCC microenvironment that determines the efficacy of immunotherapy. Journal of hepatology. 2023; 78(4):770-82.
35. Khodarev N N, Roizman B, Weichselbaum R R. Molecular pathways: interferon/stat1 pathway: role in the tumor resistance to genotoxic stress and aggressive growth. Clinical cancer research. 2012; 18(11):3015-21.
36. Jones D M, Read K A, Oestreich K J. Dynamic roles for IL-2-STAT5 signaling in effector and regulatory CD4+ T cell populations. The Journal of immunology. 2020; 205(7):1721-30.
37. Yuen V W-H, Chiu D K-C, Law C-T, Cheu J W-S, Chan C Y-K, Wong B P-Y, et al. Using mouse liver cancer models based on somatic genome editing to predict immune checkpoint inhibitor responses. Journal of Hepatology. 2023; 78(2):376-89.
38. Huffaker T B, Ekiz H A, Barba C, Lee S-H, Runtsch M C, Nelson M C, et al. A Stat1 bound enhancer promotes Nampt expression and function within tumor associated macrophages. Nature communications. 2021; 12(1):2620.
39. Travelli C, Colombo G, Mola S, Genazzani A A, Porta C. NAMPT: A pleiotropic modulator of monocytes and macrophages. Pharmacological research. 2018; 135:25-36. 40. Wu Y, Pu C, Fu Y, Dong G, Huang M, Sheng C. NAMPT-targeting PROTAC promotes antitumor immunity via suppressing myeloid-derived suppressor cell expansion. Acta Pharmaceutica Sinica B. 2022; 12(6):2859-68.
41. Ghiringhelli F, Apetoh L, Tesniere A, Aymeric L, Ma Y, Ortiz C, et al. Activation of the NLRP3 inflammasome in dendritic cells induces IL-1β-dependent adaptive immunity against tumors. Nature medicine. 2009; 15(10):1170-8.
42. Chiu D K-C, Tsc A P-W, Xu I M-J, Di Cui J, Lai R K-H, Li L L, et al. Hypoxia inducible factor HIF-1 promotes myeloid-derived suppressor cells accumulation through ENTPD2/CD39L1 in hepatocellular carcinoma. Nature communications. 2017; 8(1):517.
43. Cristescu R, Aurora-Garg D, Albright A, Xu L, Liu X Q, Loboda A, et al. Tumor mutational burden predicts the efficacy of pembrolizumab monotherapy: a pan-tumor retrospective analysis of participants with advanced solid tumors. Journal for immunotherapy of cancer. 2022; 10(1).
44. Marabelle A, Fakih M, Lopez J, Shah M, Shapira-Frommer R, Nakagawa K, et al. Association of tumour mutational burden with outcomes in patients with advanced solid tumours treated with pembrolizumab: prospective biomarker analysis of the multicohort, open-label, phase 2 KEYNOTE-158 study. The Lancet Oncology. 2020; 21(10):1353-65.
45. McGrail D J, Pilie P G, Rashid N U, Voorwerk L, Slagter M, Kok M, et al. High tumor mutation burden fails to predict immune checkpoint blockade response across all cancer types. Annals of Oncology. 2021; 32(5):661-72.
46. Jung J, Heo Y J, Park S. High tumor mutational burden predicts favorable response to anti-PD-(L) 1 therapy in patients with solid tumor: a real-world pan-tumor analysis. Journal for immunotherapy of cancer. 2023; 11(4).
47. Garon E B, Rizvi N A, Hui R, Leighl N, Balmanoukian A S, Eder J P, et al. Pembrolizumab for the treatment of non-small-cell lung cancer. New England Journal of Medicine. 2015; 372(21):2018-28.
48. Taube J M, Klein A, Brahmer J R, Xu H, Pan X, Kim J H, et al. Association of PD-1, PD-1 ligands, and other features of the tumor immune microenvironment with response to anti-PD-1 therapy. Clinical cancer research. 2014; 20(19):5064-74.
49. Davis A A, Patel V G. The role of PD-L1 expression as a predictive biomarker: an analysis of all US Food and Drug Administration (FDA) approvals of immune checkpoint inhibitors. Journal for immunotherapy of cancer. 2019; 7(1):278.
50. Topalian S L, Taube J M, Anders R A, Pardoll D M. Mechanism-driven biomarkers to guide immune checkpoint blockade in cancer therapy. Nature Reviews Cancer. 2016; 16(5):275-87.
51. Gibney G T, Weiner L M, Atkins M B. Predictive biomarkers for checkpoint inhibitor-based immunotherapy. The Lancet Oncology. 2016; 17(12):e542-e51.
52. Chen J, Jiang C C, Jin L, Zhang X D. Regulation of PD-L1: a novel role of pro-survival signalling in cancer. Annals of oncology. 2016; 27(3):409-16.
53. Mezzadra R, Sun C, Jac L T, Gomez-Eerland R, De Vries E, Wu W, et al. Identification of CMTM6 and CMTM4 as PD-L1 protein regulators. Nature. 2017; 549(7670):106-10.
54. Burr M L, Sparbier C E, Chan Y-C, Williamson J C, Woods K, Beavis P A, et al. CMTM6 maintains the expression of PD-L1 and regulates anti-tumour immunity. Nature. 2017; 549(7670):101-5.
55. Chui N N Q, Cheu J W S, Yuen V W H, Chiu D K C, Goh C C, Lee D, et al. Inhibition of CMTM4 sensitizes cholangiocarcinoma and Hepatocellular Carcinoma to T cell-mediated antitumor immunity through PD-L1. Hepatology communications. 2022; 6(1):178-93.
56. Tumeh P C, Harview C L, Yearley J H, Shintaku I P, Taylor E J M, Robert L, et al. PD-1 blockade induces responses by inhibiting adaptive immune resistance. Nature. 2014; 515(7528):568-71.
57. Chen P-L, Roh W, Reuben A, Cooper Z A, Spencer C N, Prieto P A, et al. Analysis of immune signatures in longitudinal tumor samples yields insight into biomarkers of response and mechanisms of resistance to immune checkpoint blockade. Cancer discovery. 2016; 6(8):827-37.
58. Cheu J W-S, Chiu D K-C, Kwan K K-L, Yang C, Yuen V W-H, Goh C C, et al. Hypoxia-inducible factor orchestrates adenosine metabolism to promote liver cancer development. Science Advances. 2023; 9(18): eade5111.
59. Cheu J W-S, Lee D, Li Q, Goh C C, Bao M H-R, Yuen V W-H, et al. Ferroptosis suppressor protein 1 inhibition promotes tumor ferroptosis and anti-tumor immune responses in liver cancer. Cellular and Molecular Gastroenterology and Hepatology. 2023; 16(1):133-59.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the method and compositions described herein. Such equivalents are intended to be encompassed by the following claims.

We claim:

1. A method treating a subject in need thereof, the method comprising:
(a) determining that the subject is substantially unresponsive to immune checkpoint inhibitors by determining that the subject has elevated nicotinamide adenine dinucleotide (NAD) levels relative to average NAD levels of HCC patients; and
(b) administering to the subject determined to be substantially unresponsive to immune checkpoint inhibitors an effective amount of:
(i) an inhibitor of P2X7 receptor,
(ii) an inhibitor of nicotinamide phosphoribosyltransferase (NAMPT), or
(iii) a combination thereof.

2. The method of claim 1, wherein the subject has hepatocellular carcinoma (HCC) or has been identified as being at increased risk of developing HCC.

3. The method of claim 1, wherein, prior to the administering, the subject has been determined to have HCC or to be at increased risk of developing HCC.

4. The method of claim 1, wherein the patient has elevated nicotinamide adenine dinucleotide (NAD) levels when treated with an immune checkpoint inhibitor relative to average NAD levels of HCC patients when treated with an immune checkpoint inhibitor.

5. The method of claim 1, wherein the elevated NAD levels are detected in serum and/or tissues of the subject.

6. The method of claim 1 further comprising treating the subject with an immune checkpoint inhibitor at the same time or after the administering.

7. The method of claim 6, wherein the administering is in combination with the immune checkpoint inhibitor.

8. The method of claim 6, wherein the immune checkpoint inhibitor targets programmed cell death protein 1 (PD-1), programmed cell death ligand 1 (PD-L1), cytotoxic T-lymphocyte-associated protein 4 (CTLA-4), lymphocyte-activation gene 3 (LAG-3), or combination thereof.

9. The method of claim 1, wherein the inhibitor of P2X7 receptor is a polypeptide, a small molecule, a peptidomimetic, a nucleic acid that targets genomic or expressed nucleic acids encoding the P2X7 receptor, or a vector that encodes an inhibitory nucleic acid that targets genomic or expressed nucleic acids encoding the P2X7 receptor.

10. The method of claim 9, wherein the polypeptide is an antibody.

11. The method of claim 9, wherein the expressed nucleic acid is mRNA.

12. The method of claim 1, wherein the inhibitor of P2X7 receptor is A438079, A-740003, AZ-10606120, or a derivative thereof.

13. The method of claim 1, wherein the inhibitor of NAMPT is a polypeptide, a small molecule, a peptidomimetic, a nucleic acid that targets genomic or expressed nucleic acids encoding NAMPT, or a vector that encodes an inhibitory nucleic acid that targets genomic or expressed nucleic acids encoding NAMPT.

14. The method of claim 13, wherein the polypeptide is an antibody.

15. The method of claim 13, wherein the expressed nucleic acid is mRNA.

16. The method of claim 1, wherein the NAMPT inhibitor is a functional nucleic acid selected from the group consisting of an antisense molecule, siRNA, miRNA, aptamer, ribozyme, triplex forming molecule, RNAi, and external guide sequence.

17. The method of claim 13, wherein the small molecule is FK866, GMX1778, or a derivative thereof.
